(19) United States
(12) Patent Application Publication
(10) Pub. No.: US 2021/0292333 A1
(43) Pub. Date: Sep. 23, 2021

(54) CONDENSED AZAHETEROARYL COMPOUNDS HAVING ANTIBACTERIAL ACTIVITY AGAINST TUBERCULOSIS BACTERIA

(71) Applicant: CADILA HEALTHCARE LIMITED, Ahmedabad, Gujarat (IN)

(72) Inventors: Ranjit DESAI, Ahmedabad, Gujarat (IN); Vrajesh PANDYA, Ahmedabad, Gujarat (IN); Mehul PUJARA, Ahmedabad, Gujarat (IN); Anil ARGADE, Ahmedabad, Gujarat (IN); Jignesh JOSHI, Ahmedabad, Gujarat (IN); Anshul SATYANAND, Ahmedabad, Gujarat (IN)

(21) Appl. No.: 17/252,494

(22) PCT Filed: Jun. 14, 2019
(86) PCT No.: PCT/IB2019/054997

§ 371 Date: Dec. 15, 2020

(30) Foreign Application Priority Data
Jun. 15, 2018 (IN) 201821022468
Feb. 22, 2019 (IN) 201921007053

(51) Int. Cl. C07D 487/04; C07D 471/04; A61K 45/06; A61P 31/04

(52) U.S. Cl. CPC C07D 487/04; A61P 31/04; A61K 45/06; C07D 471/04

(57) ABSTRACT

Present invention relates to novel compound of general formula (I), their enantiomers, their diastereomers, their pharmaceutically accepted salts, or pro-drugs thereof, which are useful for the treatment of bacterial infection. The compounds of general formula (I) exhibit DprE1 enzyme inhibitory activity.

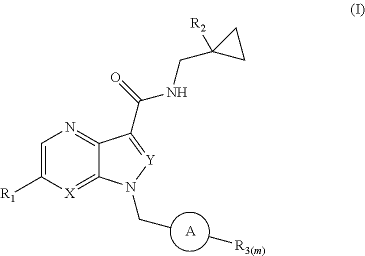

### FIELD OF INVENTION

The present invention relates to novel compounds of the general formula (I) having an excellent antibacterial activity against tuberculosis bacteria, including their tautomeric forms, their stereoisomers, their pharmaceutically acceptable salts, pharmaceutical compositions containing them, methods for their preparation, use of these compounds in medicine and the intermediates involved in their preparation. The compounds of the general formula (I) have DprE1 enzyme inhibitory activity.

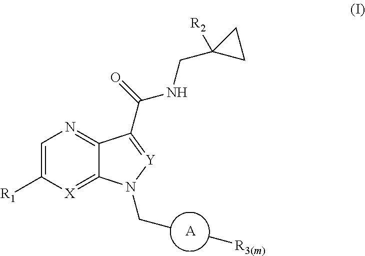

### BACKGROUND OF THE INVENTION

tuberculosis (TB) is one of the leading cause of human death from infectious disease in the world. About 33% of world's population is thought to be infected with Mycobacterium tuberculosis (Mtb), the pathogen that causes TB disease (WHO “Global tuberculosis Report 2016”, World Health Organization, 2017). Current treatment has a potential to cure drug-sensitive TB, however treatment of drug-resistant or multi drug-resistant TB (MDR-TB) is challenging and needs a two years of combination chemotherapy (WHO “Multidrug-Resistant tuberculosis (MDR-TB) 2016 Update”, World Health Organization, 2016). Resistance to current treatment for MDR-TB and extensively resistant TB (XDR-TB) highlights the need of new drugs with novel mechanism of actions.

The enzyme DprE (decaprenylphosphoryl-β-D-ribose2′-epimerase) has received significant attention as a new therapeutic strategy to tackle Mtb infection. The enzyme is composed of two proteins encoded by the dprE1 and dprE2 genes. DprE1 enzyme is the FAD-containing oxidoreductase, while DprE2 is the NADH-dependent reductase, both acts as key enzymes in the biosynthesis of decaprenylphosphoryl-o-D-arabinofuranose (DPA). DPA is a precursor of mycobacterial cell wall arabinan (Wolucka B. A. et al. FEBS J. 275, 2691-2711 (2008)). Hence, inhibition of DprE1 enzyme provides a method to inhibit bacterial cell wall biosynthesis and provides a new mechanism to identify novel drugs to treat TB. Furthermore, DprE1 enzyme has expression only in bacteria and not in human which makes it safer antibacterial target.

DprE1 target is validated by the identification of BTZ-043, a covalent inhibitor of DprE1 enzyme (Trefzer, C. et al. J. Am. Chem. Soc. 132, 13663-13665, (2010)), which has demonstrated in vivo efficacy in mouse model of TB (Makarov et al. Science, 324, 801-804, (2009)). Next generation benzothiazinone derivative PBTZ-169 has shown improved antibacterial activity than BTZ-043 (Makarov et al. EMBO Mol. Med. 6, 372-383, (2014)). WO2015009525 describes azaindole derivatives for the treatment of Mycobacterium infection or tuberculosis. WO2016031255 describes heterocyclic compounds and their use for the treatment of tuberculosis. WO2017146246 describes piperidine derivatives having antibacterial activity against Mycobacterium tuberculosis. WO2018055048 describes new series of benzothiazinone compounds for the treatment of mammalian infections.

As per WHO report (2018), 558000 people developed TB in year 2017 that was resistant to rifampicin, the most effective first line drug, and of these, 82% had multidrug-resistant TB (MDR-TB). 10% of MDR patients converted to XDR-TB (extensively drug-resistant TB). This highlights the importance of new anti-TB drugs with new mechanism of action such as DprE1 that can tackle the issue of MDR-TB.

Preclinical evaluation of TB treatment outcome occurs primarily in mouse models (The Journal of infectious diseases 211(Suppl 3), S83-95, 2015). Hence it is desirable that investigational compounds show good oral bioavailability and high blood exposure in mice. Compounds of the invention have shown excellent oral bioavailability and blood exposure in mice (Table 3) in addition to desired biological activity.

### SUMMARY OF THE INVENTION

The present invention discloses novel compounds of the general formula (I) having an excellent antibacterial activity against tuberculosis bacteria. The compounds of the present invention are useful in the treatment of the human or animal body, by regulation of DprE1 enzyme. The compounds of this invention are therefore suitable for the treatment of tuberculosis or Mycobacterium infection.

### EMBODIMENTS OF THE INVENTION

The main objective of the present invention is to provide novel compounds of general formula (I), their tautomeric forms, novel intermediates involved in their synthesis, their pharmaceutically acceptable salts, their pharmaceutically acceptable solvates and pharmaceutical compositions containing them or their mixtures suitable for the treatment of tuberculosis or Mycobacterium infection.

In an embodiment is provided a process for the preparation of novel compounds of general formula (I), their tautomeric forms, novel intermediates involved in their synthesis, their pharmaceutically acceptable salts, pharmaceutically acceptable solvates and pharmaceutical compositions containing them.

In another embodiment is provided pharmaceutical compositions containing compounds of general formula (I), their tautomeric forms, their pharmaceutically acceptable salts, solvates and their mixtures having pharmaceutically acceptable carriers, solvents, diluents, excipients and other media normally employed in their manufacture.

In a further embodiment is provided the use of the novel compounds of the present invention for the treatment of mammalian infections such as tuberculosis, by administering a therapeutically effective & non-toxic amount of the compound of formula (I), or their pharmaceutically acceptable compositions to the mammals.

In yet another embodiment is provided a pharmaceutical composition comprising the compound of formula (I) and a second suitable therapeutic agent for the treatment of mammalian infections such as tuberculosis.

### DESCRIPTION OF THE INVENTION

Accordingly, the present invention relates to compounds of the general formula (I),

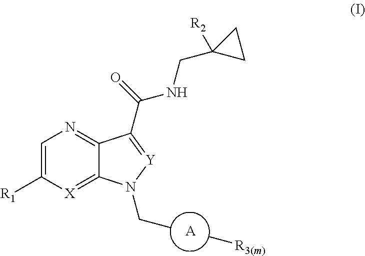

### Wherein,

“A” represents optionally substituted 6-membered ring systems as given below:

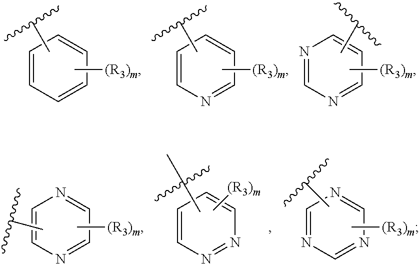

“X” and “Y” independently represents either CH or N; provided that X and Y both can not be CH together;
R₁ is independently selected from the group comprising of hydrogen, halo, haloalkyl cyano, —OR₄, —S(O)ₚR₄, —NR₄R₅, —COOR₄, optionally substituted (C₁-C₆)alkyl, (C₃-C₆)cycloalkyl, and heterocyclyl derivatives;
With a proviso that when R₁ is H, then ‘A’ represents cyclic ring other than phenyl and pyridine;
R₂ is independently selected from the group comprising of halogen, —OR₆, and —S(O)ₚR₆;
R₃ at each occurrence is independently selected from the group comprising of hydrogen, halo, cyano, haloalkyl, —NR₄R₅, S(O)ₚR₄, —OR₄, and optionally substituted (C₁-C₆)alkyl;
Each of R₄ and R₅ are at each occurrence is independently selected from hydrogen, optionally substituted (C₁-C₆)alkyl, (C₃-C₆)cycloalkyl, cycloalkanyl alkyl, heterocyclyl, haloalkyl; alternatively, R₄ and R₅ together with the N atom to which they are attached may form a 5-8 membered cyclic ring which may contain additional heteroatoms O, —S(O)ₚ and —NR₆;
R₆ is selected from the H or (C₁-C₆)alkyl;
In an embodiment, the (C₁-C₆) alkyl chain as used herein before, may further substituted with hydrogen, hydroxy, cyano, halo, haloalkyl, oxo, (C₁-C₆)alkyl, (C₃-C₆)cycloalkyl, heterocyclyl, alkylsulfonyloxy, —COOR₄, —OR₄, —S(O)ₚR₄, —NR₄R₅, —CONR₄R₅, —N(R₄)COR₅, —SO₂NR₄R₅, —N(R₄)SO₂R₅ derivatives; wherein, R₄ and R₅ are as defined earlier;
p represents integers from 0-2; m represents integers from 1-5;
Further preferred embodiments are those disclosed below.
Preferred “A” may be selected from following groups;

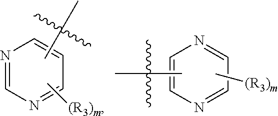

Preferred R₁ group is selected from —CH₃, —OCH₃, and halogen; Preferred R₂ group is hydroxy;
Preferred R₃ group is selected from —CH₃, —OR₄, —SCH₃, —N(CH₃)₂ and halogen, wherein R₄ is as defined earlier;
In a further embodiment the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, and the like;
the “cycloalkyl”, or “alicyclic” group used either alone or in combination with other radicals, is selected from a cyclic radical containing three to six carbons, more preferably cyclopropyl, cyclobutyl, cyclopentyl, cyclohexyl and the like;
the “haloalkyl” group is selected from an alkyl radical, as defined above, suitably substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “heterocyclyl” or “heterocyclic” group used either alone or in combination with other radicals, is selected from suitable saturated, partially saturated or unsaturated aromatic or non-aromatic mono, bi or tricyclic radicals, containing one or more heteroatoms selected from nitrogen, sulfur and oxygen, more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl, and the like; In one embodiment, the heterocycle group, wherever applicable, may consists of appropriate number of carbon atoms and include from 1-4 heteroatoms selected from the group consisting of N, O, and S(O)ₚ, p=0-2;
the “aryl” or “aromatic” group used either alone or in combination with other radicals, is selected from a suitable aromatic system such as phenyl and the like;
the “heteroaryl” or “heteroaromatic” group used either alone or in combination with other radicals, is selected from aromatic heterocyclic radicals containing one or more hetero atoms selected from N, more preferably the groups are selected from pyridyl, pyrimidinyl, pyridazinyl, triazinyl, pyrazinyl and the like;
the “cycloalkanyl alkyl” group used either alone or in combination with other radicals, is selected from groups containing an cycloalkyl radical, as defined above, attached directly to an alkyl radical, as define above;
the “alkylsulfonyloxy” group used either alone or in combination, refers to an alkylsulfonyl group attached directly to an oxygen atom, wherein a suitable alkyl group as defined above is attached to a sulfonyl radical;
the “oxo” or “carbonyl” group used either alone (—C═O—) or in combination with other radicals such as alkyl described above, for e.g. “alkylcarbonyl”, denotes a carbonyl radical (—C═O—) substituted with an alkyl radical described above such as acyl or alkanoyl;
   Suitable groups and substituents on the groups may be selected from those described anywhere in the specification.
   Preferred compounds according to the present invention include but not limited to:
N-((1-hydroxycyclopropyl)methyl)-1-((6-methoxy-5-methylpyrimidin-4-yl)methyl)-6-methyl-1H-pyrazolo[4,3-b]pyridine-3-carboxamide;
1-((6-methoxy-5-methylpyrimidin-4-yl)methyl)-N-((1-methoxycyclopropyl)methyl)-6-methyl-1H-pyrazolo[4,3-b]pyridine-3-carboxamide;
1-((6-ethoxy-5-methylpyrimidin-4-yl)methyl)-N-((1-hydroxycyclopropyl)methyl)-6-methyl-1H-pyrazolo[4,3-b]pyridine-3-carboxamide;
N-((1-hydroxycyclopropyl)methyl)-6-methyl-1-((5-methyl-6-(methylthio)pyrimidin-4-yl)methyl)-1H-pyrazolo[4,3-b]pyridine-3-carboxamide;
N-((1-hydroxycyclopropyl)methyl)-1-((6-(2-methoxyethoxy)-5-methylpyrimidin-4-yl)methyl)-6-methyl-1H-pyrazolo[4,3-b]pyridine-3-carboxamide;
1-((6-(dimethylamino)-5-methylpyrimidin-4-yl)methyl)-N-((1-hydroxycyclopropyl) methyl)-6-methyl-1H-pyrazolo[4,3-b]pyridine-3-carboxamide;
N-((1-hydroxycyclopropyl)methyl)-6-methyl-1-((5-methyl-6-morpholinopyrimidin-4-yl)methyl)-1H-pyrazolo[4,3-b]pyridine-3-carboxamide;
1-((6-(cyclopropylmethoxy)-5-methylpyrimidin-4-yl)methyl)-N-((1-hydroxycyclopropyl) methyl)-6-methyl-1H-pyrazolo[4,3-b]pyridine-3-carboxamide;
N-((1-hydroxycyclopropyl)methyl)-1-((2-methoxy-6-methylpyridin-3-yl)methyl)-6-methyl-1H-pyrazolo[4,3-b]pyridine-3-carboxamide;
1-((6-methoxy-5-methylpyrimidin-4-yl)methyl)-N-(2-methoxyethyl)-6-methyl-1H-pyrazolo[4,3-b]pyridine-3-carboxamide;
N-((1-hydroxycyclopropyl)methyl)-5-((6-methoxy-5-methylpyrimidin-4-yl)methyl)-3-methyl-5H-pyrrolo[2,3-b]pyrazine-7-carboxamide;
N-((1-hydroxycyclopropyl)methyl)-3-methoxy-5-((6-methoxy-5-methylpyrimidin-4-yl)methyl)-5H-pyrrolo[2,3-b]pyrazine-7-carboxamide;
5-((6-ethoxy-5-methylpyrimidin-4-yl)methyl)-N-((1-hydroxycyclopropyl)methyl)-3-methyl-5H-pyrrolo[2,3-b]pyrazine-7-carboxamide;
N-((1-hydroxycyclopropyl)methyl)-1-((6-methoxy-5-methylpyrimidin-4-yl)methyl)-6-methyl-1H-pyrazolo[3,4-b]pyrazine-3-carboxamide;
N-((1-hydroxycyclopropyl)methyl)-6-methoxy-1-((6-methoxy-5-methylpyrimidin-4-yl)methyl)-1H-pyrazolo[3,4-b]pyrazine-3-carboxamide;
N-((1-hydroxycyclopropyl)methyl)-6-methyl-1-((5-methyl-6-(oxetan-3-yloxy)pyrimidin-4-yl)methyl)-1H-pyrazolo[4,3-b]pyridine-3-carboxamide;
N-((1-hydroxycyclopropyl)methyl)-6-methoxy-1-((6-methoxy-5-methylpyrimidin-4-yl)methyl)-1H-pyrazolo[4,3-b]pyridine-3-carboxamide;
1-((6-ethoxy-5-methylpyrimidin-4-yl)methyl)-N-((1-hydroxycyclopropyl)methyl)-6-methoxy-1H-pyrazolo[4,3-b]pyridine-3-carboxamide;
1-((6-(dimethylamino)-5-methylpyrimidin-4-yl)methyl)-N-((1-hydroxycyclopropyl) methyl)-6-methoxy-1H-pyrazolo[4,3-b]pyridine-3-carboxamide;
1-((6-(ethylthio)-5-methylpyrimidin-4-yl)methyl)-N-((1-hydroxycyclopropyl)methyl)-6-methyl-1H-pyrazolo[4,3-b]pyridine-3-carboxamide;
1-((6-(difluoromethoxy)-5-methylpyrimidin-4-yl)methyl)-N-((1-hydroxycyclopropyl) methyl)-6-methyl-1H-pyrazolo[4,3-b]pyridine-3-carboxamide;
N-((1-fluorocyclopropyl)methyl)-1-((6-methoxy-5-methylpyrimidin-4-yl)methyl)-6-methyl-1H-pyrazolo[4,3-b]pyridine-3-carboxamide;
1-((5-fluoro-2,6-dimethylpyridin-3-yl)methyl)-N-((1-hydroxycyclopropyl)methyl)-6-methyl-1H-pyrazolo[4,3-b]pyridine-3-carboxamide;
1-((3,5-dimethylpyrazin-2-yl)methyl)-N-((1-hydroxycyclopropyl)methyl)-6-methyl-1H-pyrazolo[4,3-b]pyridine-3-carboxamide;
6-chloro-N-((1-hydroxycyclopropyl)methyl)-1-((6-methoxy-5-methylpyrimidin-4-yl)methyl)-1H-pyrazolo[4,3-b]pyridine-3-carboxamide;
1-((5-fluoro-2-methoxy-6-methylpyridin-3-yl)methyl)-N-((1-hydroxycyclopropyl) methyl)-6-methyl-1H-pyrazolo[4,3-b]pyridine-3-carboxamide;
N-((1-hydroxycyclopropyl)methyl)-1-((2-methoxy-5-(trifluoromethyl)pyridin-3-yl)methyl)-6-methyl-1H-pyrazolo[4,3-b]pyridine-3-carboxamide;
1-((5-fluoro-2-methoxypyridin-3-yl)methyl)-N-((1-hydroxycyclopropyl)methyl)-6-methyl-1H-pyrazolo[4,3-b]pyridine-3-carboxamide;
1-(2,4-dimethylbenzyl)-N-((1-hydroxycyclopropyl)methyl)-6-methyl-1H-pyrazolo[4,3-b]pyridine-3-carboxamide;
5-((6-ethoxy-5-methylpyrimidin-4-yl)methyl)-N-((1-hydroxycyclopropyl)methyl)-3-methoxy-5H-pyrrolo[2,3-b]pyrazine-7-carboxamide;
5-((6-(dimethylamino)-5-methylpyrimidin-4-yl)methyl)-N-((1-hydroxycyclopropyl) methyl)-3-methyl-5H-pyrrolo[2,3-b]pyrazine-7-carboxamide;
5-((6-(dimethylamino)-5-methylpyrimidin-4-yl)methyl)-N-((1-hydroxycyclopropyl) methyl)-3-methoxy-5H-pyrrolo[2,3-b]pyrazine-7-carboxamide;
N-((1-hydroxycyclopropyl)methyl)-3-methoxy-5-((5-methyl-6-(methylthio)pyrimidin-4-yl)methyl)-5H-pyrrolo[2,3-b]pyrazine-7-carboxamide
N-((1-hydroxycyclopropyl)methyl)-3-methyl-5-((5-methyl-6-(methylthio)pyrimidin-4-yl)methyl)-5H-pyrrolo[2,3-b]pyrazine-7-carboxamide;
5-((6-(ethylthio)-5-methylpyrimidin-4-yl)methyl)-N-((1-hydroxycyclopropyl)methyl)-3-methyl-5H-pyrrolo[2,3-b]pyrazine-7-carboxamide
5-((6-(ethylthio)-5-methylpyrimidin-4-yl)methyl)-N-((1-hydroxycyclopropyl)methyl)-3-methoxy-5H-pyrrolo[2,3-b]pyrazine-7-carboxamide;
5-((6-(difluoromethoxy)-5-methylpyrimidin-4-yl)methyl)-N-((1-hydroxycyclopropyl) methyl)-3-methyl-5H-pyrrolo[2,3-b]pyrazine-7-carboxamide;
5-((6-(difluoromethoxy)-5-methylpyrimidin-4-yl)methyl)-N-((1-hydroxycyclopropyl) methyl)-3-methoxy-5H-pyrrolo[2,3-b]pyrazine-7-carboxamide;
N-((1-fluorocyclopropyl)methyl)-5-((6-methoxy-5-methylpyrimidin-4-yl)methyl)-3-methyl-5H-pyrrolo[2,3-b]pyrazine-7-carboxamide;
N-((1-fluorocyclopropyl)methyl)-3-methoxy-5-((6-methoxy-5-methylpyrimidin-4-yl)methyl)-5H-pyrrolo[2,3-b]pyrazine-7-carboxamide;
N-((1-hydroxycyclopropyl)methyl)-5-((2-methoxy-6-methylpyridin-3-yl)methyl)-3-methyl-5H-pyrrolo[2,3-b]pyrazine-7-carboxamide;
N-((1-hydroxycyclopropyl)methyl)-3-methoxy-5-((2-methoxy-6-methylpyridin-3-yl)methyl)-5H-pyrrolo[2,3-b]pyrazine-7-carboxamide;
5-((5-fluoro-2,6-dimethylpyridin-3-yl)methyl)-N-((1-hydroxycyclopropyl)methyl)-3-methyl-5H-pyrrolo[2,3-b]pyrazine-7-carboxamide;
5-((5-fluoro-2,6-dimethylpyridin-3-yl)methyl)-N-((1-hydroxycyclopropyl)methyl)-3-methoxy-5H-pyrrolo[2,3-b]pyrazine-7-carboxamide;
5-((3,5-dimethylpyrazin-2-yl)methyl)-N-((1-hydroxycyclopropyl)methyl)-3-methyl-5H-pyrrolo[2,3-b]pyrazine-7-carboxamide;
5-((3,5-dimethylpyrazin-2-yl)methyl)-N-((1-hydroxycyclopropyl)methyl)-3-methoxy-5H-pyrrolo[2,3-b]pyrazine-7-carboxamide;
N-((1-hydroxycyclopropyl)methyl)-5-((6-methoxy-5-methylpyrimidin-4-yl)methyl)-5H-pyrrolo[2,3-b]pyrazine-7-carboxamide;
5-((5-fluoro-2-methoxy-6-methylpyridin-3-yl)methyl)-N-((1-hydroxycyclopropyl) methyl)-3-methyl-5H-pyrrolo[2,3-b]pyrazine-7-carboxamide;
5-((5-fluoro-2-methoxy-6-methylpyridin-3-yl)methyl)-N-((1-hydroxycyclopropyl) methyl)-3-methoxy-5H-pyrrolo[2,3-b]pyrazine-7-carboxamide;
5-((5-fluoro-2-methoxypyridin-3-yl)methyl)-N-((1-hydroxycyclopropyl)methyl)-3-methyl-5H-pyrrolo[2,3-b]pyrazine-7-carboxamide;
5-((5-fluoro-2-methoxypyridin-3-yl)methyl)-N-((1-hydroxycyclopropyl)methyl)-3-methoxy-5H-pyrrolo[2,3-b]pyrazine-7-carboxamide;
N-((1-hydroxycyclopropyl)methyl)-5-((2-methoxy-5-(trifluoromethyl)pyridin-3-yl)methyl)-3-methyl-5H-pyrrolo[2,3-b]pyrazine-7-carboxamide;
N-((1-hydroxycyclopropyl)methyl)-3-methoxy-5-((2-methoxy-5-(trifluoromethyl)pyridin-3-yl)methyl)-5H-pyrrolo[2,3-b]pyrazine-7-carboxamide;
3-(dimethylamino)-N-((1-hydroxycyclopropyl)methyl)-5-((6-methoxy-5-methylpyrimidin-4-yl)methyl)-5H-pyrrolo[2,3-b]pyrazine-7-carboxamide;
3-cyano-N-((1-hydroxycyclopropyl)methyl)-5-((6-methoxy-5-methylpyrimidin-4-yl)methyl)-5H-pyrrolo[2,3-b]pyrazine-7-carboxamide;
3-ethoxy-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-3-isopropoxy-5-((6-methoxy-5-methylpyrimidin-4-yl)methyl)-5H-pyrrolo[2,3-b]pyrazine-7-carboxamide;
N-((1-hydroxycyclopropyl)methyl)-6-methyl-1-((5-methyl-6-(methylthio)pyrimidin-4-yl)methyl)-1H-pyrazolo[3,4-b]pyrazine-3-carboxamide;
N-((1-hydroxycyclopropyl)methyl)-6-methoxy-1-((5-methyl-6-(methylthio)pyrimidin-4-yl)methyl)-1H-pyrazolo[3,4-b]pyrazine-3-carboxamide;
1-((6-(dimethylamino)-5-methylpyrimidin-4-yl)methyl)-N-((1-hydroxycyclopropyl) methyl)-6-methoxy-1H-pyrazolo[3,4-b]pyrazine-3-carboxamide;
1-((6-(dimethylamino)-5-methylpyrimidin-4-yl)methyl)-N-((1-hydroxycyclopropyl) methyl)-6-methyl-1H-pyrazolo[3,4-b]pyrazine-3-carboxamide;
1-((6-ethoxy-5-methylpyrimidin-4-yl)methyl)-N-((1-hydroxycyclopropyl)methyl)-6-methyl-1H-pyrazolo[3,4-b]pyrazine-3-carboxamide;
1-((6-ethoxy-5-methylpyrimidin-4-yl)methyl)-N-((1-hydroxycyclopropyl)methyl)-6-methoxy-1H-pyrazolo[3,4-b]pyrazine-3-carboxamide;
1-((6-(difluoromethoxy)-5-methylpyrimidin-4-yl)methyl)-N-((1-hydroxycyclopropyl) methyl)-6-methyl-1H-pyrazolo[3,4-b]pyrazine-3-carboxamide;
1-((6-(difluoromethoxy)-5-methylpyrimidin-4-yl)methyl)-N-((1-hydroxycyclopropyl) methyl)-6-methoxy-1H-pyrazolo[3,4-b]pyrazine-3-carboxamide;
N-((1-hydroxycyclopropyl)methyl)-1-((2-methoxy-6-methylpyridin-3-yl)methyl)-6-methyl-1H-pyrazolo[3,4-b]pyrazine-3-carboxamide;
N-((1-hydroxycyclopropyl)methyl)-6-methoxy-1-((2-methoxy-6-methylpyridin-3-yl)methyl)-1H-pyrazolo[3,4-b]pyrazine-3-carboxamide;
1-((5-fluoro-2,6-dimethylpyridin-3-yl)methyl)-N-((1-hydroxycyclopropyl)methyl)-6-methyl-1H-pyrazolo[3,4-b]pyrazine-3-carboxamide;
1-((5-fluoro-2,6-dimethylpyridin-3-yl)methyl)-N-((1-hydroxycyclopropyl)methyl)-6-methoxy-1H-pyrazolo[3,4-b]pyrazine-3-carboxamide;
1-((3,5-dimethylpyrazin-2-yl)methyl)-N-((1-hydroxycyclopropyl)methyl)-6-methyl-1H-pyrazolo[3,4-b]pyrazine-3-carboxamide;
1-((3,5-dimethylpyrazin-2-yl)methyl)-N-((1-hydroxycyclopropyl)methyl)-6-methoxy-1H-pyrazolo[3,4-b]pyrazine-3-carboxamide;
N-((1-fluorocyclopropyl)methyl)-1-((6-methoxy-5-methylpyrimidin-4-yl)methyl)-6-methyl-1H-pyrazolo[3,4-b]pyrazine-3-carboxamide;
N-((1-fluorocyclopropyl)methyl)-6-methoxy-1-((6-methoxy-5-methylpyrimidin-4-yl)methyl)-1H-pyrazolo[3,4-b]pyrazine-3-carboxamide;
1-((5-fluoro-2-methoxy-6-methylpyridin-3-yl)methyl)-N-((1-hydroxycyclopropyl) methyl)-6-methyl-1H-pyrazolo[3,4-b]pyrazine-3-carboxamide;
1-((5-fluoro-2-methoxy-6-methylpyridin-3-yl)methyl)-N-((1-hydroxycyclopropyl) methyl)-6-methoxy-1H-pyrazolo[3,4-b]pyrazine-3-carboxamide;
1-((5-fluoro-2-methoxypyridin-3-yl)methyl)-N-((1-hydroxycyclopropyl)methyl)-6-methyl-1H-pyrazolo[3,4-b]pyrazine-3-carboxamide;
1-((5-fluoro-2-methoxypyridin-3-yl)methyl)-N-((1-hydroxycyclopropyl)methyl)-6-methoxy-1H-pyrazolo[3,4-b]pyrazine-3-carboxamide;
N-((1-hydroxycyclopropyl)methyl)-1-((2-methoxy-5-(trifluoromethyl)pyridin-3-yl)methyl)-6-methyl-1H-pyrazolo[3,4-b]pyrazine-3-carboxamide;
N-((1-hydroxycyclopropyl)methyl)-6-methoxy-1-((2-methoxy-5-(trifluoromethyl)pyridin-3-yl)methyl)-1H-pyrazolo[3,4-b]pyrazine-3-carboxamide;
N-((1-hydroxycyclopropyl)methyl)-6-methoxy-1-((6-methoxy-5-methylpyrimidin-4-yl)methyl)-1H-pyrrolo[3,2-b]pyridine-3-carboxamide;
1-((6-(dimethylamino)-5-methylpyrimidin-4-yl)methyl)-N-((1-hydroxycyclopropyl)methyl)-6-methoxy-1H-pyrrolo[3,2-b]pyridine-3-carboxamide;
1-((6-(dimethylamino)-5-methylpyrimidin-4-yl)methyl)-N-((1-hydroxycyclopropyl)methyl)-6-methyl-1H-pyrrolo[3,2-b]pyridine-3-carboxamide;
1-((6-ethoxy-5-methylpyrimidin-4-yl)methyl)-N-((1-hydroxycyclopropyl)methyl)-6-methyl-1H-pyrrolo[3,2-b]pyridine-3-carboxamide;
1-((6-ethoxy-5-methylpyrimidin-4-yl)methyl)-N-((1-hydroxycyclopropyl)methyl)-6-methoxy-1H-pyrrolo[3,2-b]pyridine-3-carboxamide;
N-((1-hydroxycyclopropyl)methyl)-1-((6-methoxy-5-methylpyrimidin-4-yl)methyl)-6-methyl-1H-pyrrolo[3,2-b]pyridine-3-carboxamide.
N-((1-hydroxycyclopropyl)methyl)-5-((6-methoxy-5-methylpyrimidin-4-yl)methyl)-3-(methylthio)-5H-pyrrolo[2,3-b]pyrazine-7-carboxamide;
3-cyclopropyl-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yloxy)-5H-pyrrolo[2,3-b]pyrazine-7-carboxamide;
3-(cyclopropylmethoxy)-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(2-methoxyethoxy)-5H-pyrrolo[2,3-b]pyrazine-7-carboxamide;
5-((6-methoxy-5-methylpyrimidin-4-yl)methyl)-3-methyl-N-((1-(methylthio)cyclopropyl)methyl)-5H-pyrrolo[2,3-b]pyrazine-7-carboxamide;
N-((1-hydroxycyclopropyl)methyl)-5-((6-methoxy-4-methylpyridazin-3-yl)methyl)-3-methyl-5H-pyrrolo[2,3-b]pyrazine-7-carboxamide;
5-((4-(dimethylamino)-6-methyl-1,3,5-triazin-2-yl)methyl)-N-((1-hydroxycyclopropyl)methyl)-3-methyl-5H-pyrrolo[2,3-b]pyrazine-7-carboxamide;

The novel compounds of this invention may be prepared using the reactions and techniques as shown in scheme below and described in this section. The reactions are performed in solvents appropriate to the reagents and materials employed and are suitable for the transformations being affected. It is understood by those skilled in the art that the nature and order of the synthetic steps presented may be varied for the purpose of optimizing the formation of the compounds of the present invention. It will also be well appreciated that one or more of the reactants may be protected and deprotected for facile synthesis by techniques known to persons skilled in the art. It will also be appreciated that one or more of the compounds of the present invention may exist in stereoisomeric and/or diastereomeric forms. Such stereoisomers and/or diastereoisomers as well as their optical antipodes are to be construed to be within the scope of the present invention. It will also be well appreciated that one or more of these compounds may be converted to their salts and other derivatives based on the specific groups present on the compounds, which can be well comprehended by persons skilled in the art. Such salts and/or other derivatives, as the case may be should also be construed to be within the scope of the present invention.

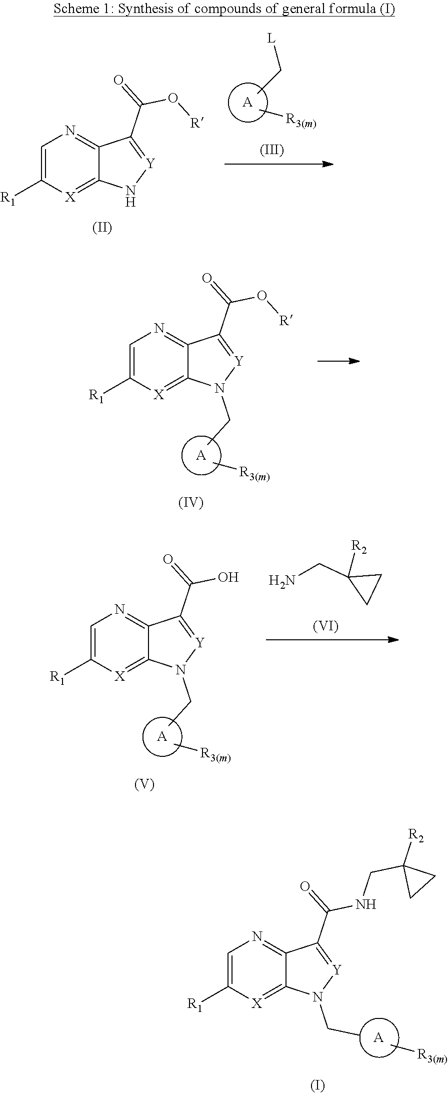

Where R′ is alkyl group such as methyl, ethyl etc.
L=suitable leaving group such as Cl, Br, I, mesyl etc.
The compounds of the general formula (II) can be prepared as described in the literature (Faming Zhuanli Shenqing, 102911174, (2013), WO201481732, WO2017120429 and WO2018049271) using appropriate modifications well known to a skilled person. The compound (IV) can be obtained by reacting compounds of the formula (II) with (III) in the presence of base such as sodium carbonate, potassium carbonate, sodium hydride etc. in solvents such as THF, DMF, methanol etc. The compounds of general formula (V) can be obtained from (IV) by hydrolysis of ester group using inorganic bases such as LiOH, NaOH, KOH etc. in solvent(s) such as water, THF, methanol or mixture thereof. The compounds of the general formula (I) are then obtained by reacting (V) with amines of general formula (VI) using various amide bond formation techniques as described for e.g. in Tetrahedron 2005, 61, 10827.
The invention is explained in further details by the examples given below, which are provided by way of illustration only and therefore should not be construed to limit the scope of the invention.
The ¹H NMR spectra were recorded on a Brucker Avance-400 spectrometer (400 MHz). The chemical shifts (δ) are reported in parts per million (ppm) relative to Tetramethyl silane (TMS), in either CDCl₃ or DMSO-d₆ solution. Mass spectra (ESI-MS) were obtained on Shimadzu LC-MS 2010-A spectrometer.

### List of Abbreviations

### CBr₄: Tetrabromomethane

CDCl₃: Deuterated chloroform

### CHCl₃: Chloroform

### CH₃OH: Methanol

DIPEA: Disopropyl ethyl amine

### EtOH: Ethanol

DMF: Dimethyl formamide

### DCM: Dichloromethane

DMF-DMA: N,N-Dimethylformamide dimethyl acetal
DMSO: Dimethyl sulfoxide
DMSO-d₆: Hexadeuterodimethyl sulfoxide
EDC.HCl: N-(3-Dimethyl aminopropyl)-N′-ethyl carbodiimide hydrochloride
HOBT: 1-Hydroxy benzotriazole
HCl: Hydrochloric acid
K₂CO₃: Potassium carbonate
LiAlH₄: Lithium aluminum hydrid
Na₂SO₄: Sodium sulfate

### NaH: Sodium Hydride

NaHCO₃: Sodium bicarbonate
NaNO₂: Sodium nitrite
NaOH: Sodium hydroxide
NaOEt: Sodium ethoxide
NaOCH₃: Sodium methoxide
NH₄Cl: Ammonium chloride
NMP: N-methyl-2-pyrrolidone
Pd/C: Palladium charcoal

### Pd(dppf)Cl₂: [1,1′-Bis(diphenylphosphino)ferrocene]dichloropalladium(II)

POCl₃: Phosphoryl chloride

### THF: Tetrahydrofuran

TPP: Triphenyl phosphine

### ¹H NMR: Proton Nuclear Magnetic Resonance

### h: Hour(s)

### min: Minute(s)

J: Coupling constant in units of Hz

### Hz: Hertz

### MABA: Microplate Alamar Blue Assay

### Preparation of Compounds

### Example 1

### Preparation of N-((1-hydroxycyclopropyl)methyl)-1-((6-methoxy-5-methylpyrimidin-4-yl)methyl)-6-methyl-1H-pyrazolo[4,3-b]pyridine-3-carboxamide Preparation of ethyl 6-methyl-1H-pyrazolo[4,3-b]pyridine-3-carboxylate (Intermediate 1A)

### Step 1: 1-(tert-butyl) 3-ethyl 2-(5-methyl-3-nitropyridin-2-yl)malonate

To a stirring suspension of 60% NaH (3.5 g, 87 mmol) in dry DMF (100 ml) was added tert-butyl ethyl malonate (14.18 g, 75 mmol) drop wise over a period of 15 min. To this was added 2-chloro-5-methyl-3-nitropyridine (10 g, 57.9 mmol) portion wise over a period of 15 min. and the resulting mixture was stirred at 25-30° C. for 6 h. After complete conversion of starting material reaction mixture was cooled to 15° C. The reaction mixture was diluted with water (500 ml) and extracted by ethyl acetate (2×200 ml). Organic layer was separated, washed with water, dried over Na₂SO₄ and evaporated under reduced pressure to get crude product which was further purified by column chromatography (0-40% Ethyl acetate in Hexane) to get the 12.3 g of the title product. ESI-MS (m/z): 323.04 (M−H).

### Step 2: ethyl 2-(5-methyl-3-nitropyridin-2-yl)acetate

To a stirring solution of product of step 1 (12.2 g, 37.6 mmol) in DCM (250 ml) was added trifluoro acetic acid (29 ml, 376 mmol) at 10-15° C. The reaction mixture was stirred at 25-30° C. for 10 h. After complete conversion of starting material, reaction mixture was cooled to 10° C. and washed with saturated solution of NaHCO₃ (150 ml). Organic layer was separated, washed with water, dried over Na₂SO₄ and evaporated under reduced pressure to get the 7.5 g of the title product. ESI-MS (m/z): 225.18 (M+H)⁺.

### Step 3: ethyl 2-(3-amino-5-methylpyridin-2-yl)acetate

To a stirring solution of product of step 2 (7.5 g, 33.5 mmol) in CH₃OH (75 ml) was added Pd/C (10%) (0.712 g, 6.69 mmol). The reaction mixture was stirred at 25-30° C. under H₂ pressure for 3 h. After complete conversion of starting material reaction mixture was filtered through Hyflo. The filtrate was evaporated to get the 5.1 g of the title product. ESI-MS (m/z): 195.19 (M+H)⁺.

### Step 4: ethyl 6-methyl-1H-pyrazolo[4,3-b]pyridine-3-carboxylate

To a stirring solution of product of step 3 (5.0 g, 25.7 mmol) in Conc. HCl (50 ml) was added a solution of NaNO₂ (3.0 g (48.3 mmol) in 10 ml water) at 0-5° C. over a period of 30 min. Reaction mixture was stirred at 25-30° C. for 30 min. The pH of the reaction mixture was adjusted to 7 by using 10% NaOH solution. Stirring was continued for further 60 min. at 25-30° C. After complete conversion of starting material, reaction mixture was diluted with 50 ml water and extracted with ethyl acetate. Organic layer was separated, washed with water, dried over Na₂SO₄ and evaporated under reduced pressure to get the crude product, which was purified by column chromatography (0-30% Ethyl acetate in Hexane) to get 2.2 g of the title product. ESI-MS (m/z): 206.17 (M+H)⁺.

### Preparation of 4-(chloromethyl)-6-methoxy-5-methylpyrimidine (Intermediate 1B)

### Step 1: diethyl 2-methyl-3-oxosuccinate

To a solution of diethyl oxalate (100 g, 684 mmol) and ethyl propionate (69.9 g, 684 mmol) in 600 ml of anhydrous EtOH was added NaOEt (69.8 g, 1026 mmol) at room temperature. The mixture was stirred overnight. After cooling, the mixture was adjusted to pH=7 with 6N HCl. The residue was diluted with water, and then extracted with DCM. The combined organic layers were dried over Na₂SO₄ and concentrated under vacuum to get the title product as a red liquid.

### Step 2: ethyl 6-hydroxy-5-methylpyrimidine-4-carboxylate

To a stirring solution of product of step 1 (92 g, 455 mmol) and formamidine acetate (71.1 g, 682 mmol) in EtOH (700 mL) was added NaOEt (46.40 g, 682 mmol) and reaction mixture was heated to reflux for 12 h. After cooling, the reaction mixture was adjusted to 15 pH=7 with 6N HCl. The mixture was concentrated under vacuum. The residue was diluted with water, and then extracted with DCM. The combined DCM layers were washed with water, and evaporated to get crude product. The crude product was stirred with 200 ml n-Hexane for 30 min. and filtered to get yellowish solid as a product. ESI-MS (m/z): 183.05 (M+H)⁺.

### Step 3: ethyl 6-chloro-5-methylpyrimidine-4-carboxylate

Stirring solution of product of step 2 (29.0 g, 455 mmol) in POCl₃ (290 ml) was heated to reflux for 4 hours. After cooling, the excess POCl₃ was removed under reduced pressure and the residue was diluted with cold water (500 ml) and extracted by n-Hexane. Organic layer was separated, washed with water, dried over Na₂SO₄ and evaporated under reduced pressure to get title product. ESI-MS (m/z): 201.02 (M+H)⁺.

### Step 4: ethyl 6-methoxy-5-methylpyrimidine-4-carboxylate

To a stirring solution of product of step 3 (7.0 g, 38.4 mmol) in dry CH₃OH (70 ml) was added NaOCH₃ (2.7 g, 50.0 mmol) at 0-5° C. The resulting mixture was stirred at 25-30° C. for 3 h. After complete conversion of starting material reaction mixture was cooled to 15° C. The reaction mixture was diluted with cold water (25 ml) and extracted by ethyl acetate. Organic layer was separated, washed with water, dried over Na₂SO₄ and evaporated under reduced pressure to get crude product which was used without further purification. ESI-MS (m/z): 197.08 (M+H)⁺.

### Step 5: (6-methoxy-5-methylpyrimidin-4-yl)methanol

To a stirring solution of product of step 4 (5.6 g, 28.5 mmol) in CH₃OH (56 ml) was added Sodium borohydride (1.62 g, 42.8 mmol) portion wise over a period of 10 min. at 25-30° C. The reaction mixture was stirred for 3 h. After complete conversion of starting material reaction mixture was cooled to 10° C. and was diluted with cold water (100 ml) and extracted by ethyl acetate. Organic layer was separated, washed with water, dried over Na₂SO₄ and evaporated under reduced pressure to get crude product which was used without further purification. ESI-MS (m/z): 155.30 (M+H)⁺.

### Step 6: 4-(chloromethyl)-6-methoxy-5-methylpyrimidine

To a stirring solution of product of step 5 (4.0 g, 25.9 mmol) in DCM (40 ml) was added Thionyl chloride (3.80 ml, 51.9 mmol) at 5-10° C. The reaction mixture was stirred at 25-30° C. for 1 h. After complete conversion of starting material, solvent was evaporated under reduced pressure to get the residue. 50 ml DCM was added to the residue and evaporated the solvent to get yellowish solid as title product. ESI-MS (m/z): 173.04 (M+H)⁺.

### Preparation of N-((1-hydroxycyclopropyl)methyl)-1-((6-methoxy-5-methylpyrimidin-4-yl)methyl)-6-methyl-1H-pyrazolo[4,3-b]pyridine-3-carboxamide

### Step 1: ethyl 1-((6-methoxy-5-methylpyrimidin-4-yl)methyl)-6-methyl-1H-pyrazolo[4,3-b]pyridine-3-carboxylate

To a stirring suspension of product of intermediate 1A (0.500 g, 2.43 mmol) and K₂CO₃ (0.505 g, 3.65 mmol) in dry DMF (5 ml) was added intermediate 1B (0.505 g, 2.92 mmol) at 5-10° C. and the resulting mixture was stirred at 25-30° C. for 3 h. After complete conversion of starting material reaction mixture was diluted with water (20 ml) and extracted by ethyl acetate. Organic layer was separated, washed with water, dried over Na₂SO₄ and evaporated under reduced pressure to get the crude product which was further purified by column chromatography (0-20% Ethyl acetate in Hexane) to get the title product. ESI-MS (m/z): 342.15 (M+H)⁺.

### Step 2: 1-((6-methoxy-5-methylpyrimidin-4-yl)methyl)-6-methyl-1H-pyrazolo[4,3-b]pyridine-3-carboxylic acid

To a stirring solution of product of step 1 (0.5 g, 1.46 mmol) prepared above in EtOH (4 ml) was added NaOH (90 mg (2.19 mmol) in 1 ml water). The reaction mixture was stirred at 25-30° C. for 1 h. After complete conversion of starting material reaction mixture was diluted with water (5 ml) and acidified by using dil. HCl. The reaction mixture was extracted by ethyl acetate (2×10 ml). Organic layer was separated, washed with water, dried over Na₂SO₄ and evaporated under reduced pressure to get the title product. ESI-MS (m/z): 314.10 (M+H)⁺.

### Step 3: Preparation of N-((1-hydroxycyclopropyl)methyl)-1-((6-methoxy-5-methylpyrimidin-4-yl)methyl)-6-methyl-1H-pyrazolo[4,3-b]pyridine-3-carboxamide

To a stirring solution of product of step 2 (150 mg, 0.479 mmol) in DCM (3 mL) was added EDC.HCl (184 mg, 0.958 mmol) and HOBT (147 mg, 0.958 mmol) at 25-30° C. Reaction mixture was stirred for 10 min. at 25-30° C. To this was added 1-(aminomethyl)cyclopropanol (83 mg, 0.958 mmol) and stirred for 16 h. After 16 h, reaction mixture was diluted with DCM (10 ml). Reaction mixture was then washed with saturated solution of NaHCO₃, dried over Na₂SO₄, and filtered. Removal of the solvent under reduced pressure to get the product as a yellow solid, which was further purified by column chromatography (0-60% Ethyl acetate in Hexane) to get title product. ¹H NMR (DMSO-d₆): 8.88-8.85 (t, J=5.6 Hz, 1H), 8.59-8.58 (d, J=1.2 Hz, 1H), 8.40 (s, 1H), 8.07 (s, 1H), 5.89 (s, 2H), 5.52 (s, 1H), 3.94 (s, 3H), 3.54-3.53 (d, J=5.6 Hz, 2H), 2.51 (s, 3H), 2.25 (s, 3H), 0.63-0.56 (m, 4H). ESI-MS (m/z): 383.18 (M+H)⁺.

### Example 2

### Preparation of 1-((6-methoxy-5-methylpyrimidin-4-yl)methyl)-N-((1-methoxycyclopropyl)methyl)-6-methyl-1H-pyrazolo[4,3-b]pyridine-3-carboxamide

To a stirring solution of 1-((6-methoxy-5-methylpyrimidin-4-yl)methyl)-6-methyl-1H-pyrazolo[4,3-b]pyridine-3-carboxylic acid (150 mg, 0.479 mmol) in DCM (3 mL) was added EDC.HCl (184 mg, 0.958 mmol), HOBT (147 mg, 0.958 mmol) and DIPEA (124 mg, 0.958 mmol) at 25-30° C. Reaction mixture was stirred for 10 min. at 25-30° C. To this was added (1-methoxycyclopropyl)methanamine (97 mg, 0.958 mmol) and stirred for 16 h. After 16 h, reaction mixture was diluted with DCM (10 ml). Then the reaction mixture was washed with saturated solution of NaHCO₃, dried over Na₂SO₄, and filtered. Removal of the solvent under reduced pressure gave crude product, which was further purified by column chromatography (0-60% Ethyl acetate in Hexane) to get title product. ¹H NMR (CDCl₃): 8.99-8.97 (t, J=5.6 Hz, 1H), 8.55 (s, 1H), 8.518-8.514 (d, J=1.6 Hz, 1H), 7.74 (s, 1H), 5.71 (s, 2H), 3.99 (s, 3H), 3.81-3.79 (d, J=5.6 Hz, 2H), 3.42 (s, 3H), 2.52 (s, 3H), 2.27 (s, 3H), 0.91-0.88 (m, 2H), 0.74-0.71 (m, 2H). ESI-MS (m/z): 397.13 (M+H)⁺.

### Example 3

### Preparation of 1-((6-ethoxy-5-methylpyrimidin-4-yl)methyl)-N-((1-hydroxycyclopropyl)methyl)-6-methyl-1H-pyrazolo[4,3-b]pyridine-3-carboxamide

### Preparation of 4-(chloromethyl)-6-ethoxy-5-methylpyrimidine (Intermediate 3B)

### Step 1: ethyl 6-ethoxy-5-methylpyrimidine-4-carboxylate

To a stirring solution of ethyl 6-chloro-5-methylpyrimidine-4-carboxylate (7.0 g, 38.4 mmol) in dry EtOH (70 ml) was added NaOEt (3.4 g, 50.0 mmol) at 0-5° C. The resulting mixture was stirred at 25-30° C. for 3 h. After complete conversion of starting material reaction mixture was cooled to 15° C. The reaction mixture was diluted with cold water and extracted by ethyl acetate. Organic layer was separated, washed with water, dried over Na₂SO₄ and evaporated under reduced pressure to get crude product which was used without further purification. ESI-MS (m/z): 211.15 (M+H)⁺.

### Step 2: (6-ethoxy-5-methylpyrimidin-4-yl)methanol

To a stirring solution of product of step 1 (5.4 g, 25.7 mmol) in EtOH (54 ml) was added Sodium borohydride (1.45 g, 38.5 mmol) portion wise over a period of 10 min. at 25-30° C. The reaction mixture was stirred at 25-30° C. for 3 h. After complete conversion of starting material reaction mixture was cooled to 10° C. Reaction mixture was diluted with cold water) and extracted by ethyl acetate). Organic layer was separated, washed with water, dried over Na₂SO₄ and evaporated under reduced pressure to get crude product which was used without further purification. ESI-MS (m/z): 169.09 (M+H)⁺.

### Step 3: 4-(chloromethyl)-6-ethoxy-5-methylpyrimidine

To a stirring solution of product of step 2 (4.0 g, 23.78 mmol) in DCM (40 ml) was added Thionyl chloride (3.50 ml, 47.6 mmol) at 5-10° C. The reaction mixture was stirred at 25-30° C. for 1 h. After complete conversion of starting material, solvent was evaporated under reduced pressure to get the residue. 50 ml DCM was added to the residue and evaporated the solvent to get yellowish solid as title product. ESI-MS (m/z): 187.20 (M+H)⁺.

### Preparation of 1-((6-ethoxy-5-methylpyrimidin-4-yl)methyl)-N-((1-hydroxycyclopropyl)methyl)-6-methyl-1H-pyrazolo[4,3-b]pyridine-3-carboxamide

### Step 1: ethyl 1-((6-ethoxy-5-methylpyrimidin-4-yl)methyl)-6-methyl-1H-pyrazolo[4,3-b]pyridine-3-carboxylate

To a stirring suspension of ethyl 6-methyl-1H-pyrazolo[4,3-b]pyridine-3-carboxylate (0.200 g, 0.975 mmol) and K₂CO₃ (0.229 g, 1.65 mmol) in dry DMF (2 ml) was added Intermediate 3B (0.273 g, 1.46 mmol) at 5-10° C. The resulting mixture was stirred at 25-30° C. for 3 h. After complete conversion of starting material reaction mixture was diluted with water and extracted by ethyl acetate. Organic layer was separated, washed with water, dried over Na₂SO₄ and evaporated under reduced pressure to get the crude product which was further purified by column chromatography (0-20% Ethyl acetate in Hexane) to get the title product. ESI-MS (m/z): 356.21 (M+H)⁺.

### Step 2: 1-((6-ethoxy-5-methylpyrimidin-4-yl)methyl)-6-methyl-1H-pyrazolo[4,3-b]pyridine-3-carboxylic acid

To a stirring solution of product of step 1 (0.16 g, 0.45 mmol) in Ethanol (2 ml) was added NaOH (27 mg (0.675 mmol) in 0.5 ml water). The reaction mixture was stirred at 25-30° C. for 1 h. After complete conversion of starting material reaction mixture was diluted with water and acidified by using dil. HCl. The reaction mixture was extracted with DCM. Organic layer was separated, washed with water, dried over Na₂SO₄ and evaporated under reduced pressure to get the title product. ESI-MS (m/z): 328.10 (M+H)⁺.

### Step 3: 1-((6-ethoxy-5-methylpyrimidin-4-yl)methyl)-N-((1-hydroxycyclopropyl) methyl)-6-methyl-1H-pyrazolo[4,3-b]pyridine-3-carboxamide

To a stirring solution of product of step 2 (100 mg, 0.305 mmol) in DCM (3 mL) was added EDC.HCl (100 mg, 0.519 mmol) followed by HOBT (70 mg, 0.158 mmol) and DIPEA (80 mg, 0.611 mmol) at 25-30° C. Reaction mixture was stirred for 10 min. at 25-30° C. To this was added 1-(aminomethyl)cyclopropanol (53 mg, 0.611 mmol) and stirred for 16 h. After 16 h, reaction mixture was diluted with DCM (10 ml). Reaction mixture was then washed with saturated solution of NaHCO₃, dried over Na₂SO₄, and filtered. Removal of the solvent under reduced pressure gave product as a yellow solid, which was further purified by column chromatography (0-60% Ethyl acetate in Hexane) to get the title product. ¹H NMR (CDCl₃): 9.09-9.07 (m, 1H), 8.52 (s, 1H), 8.49-8.48 (d, J=1.2 Hz, 1H), 7.79 (s, 1H), 5.71 (s, 2H), 4.45-4.40 (m, 2H), 4.09 (s, 1H), 3.75-3.74 (d, J=6.0 Hz, 2H), 2.54 (s, 3H), 2.29 (s, 3H), 1.42-1.39 (t, J=7.2 Hz, 3H), 0.91-0.88 (m, 2H), 0.73-0.70 (m, 2H). ESI-MS (m/z): 397.19 (M+H)⁺.

### Example 4

### Preparation of N-((1-hydroxycyclopropyl)methyl)-6-methyl-1-((5-methyl-6-(methylthio)pyrimidin-4-yl)methyl)-1H-pyrazolo[4,3-b]pyridine-3-carboxamide Preparation of 4-(bromomethyl)-5-methyl-6-(methylthio)pyrimidine (Intermediate 4B)

### Step 1: ethyl 5-methyl-6-(methylthio)pyrimidine-4-carboxylate

To a stirring solution of ethyl 6-chloro-5-methylpyrimidine-4-carboxylate (2 g, 9.97 mmol) in dry DMF (10 ml) was added Sodium thiomethoxide (1.4 g, 19.94 mmol) at 0-5° C. The resulting mixture was stirred at 25-30° C. for 3 h. After complete conversion of starting material reaction mixture was cooled to 15° C. The reaction mixture was diluted with cold water (25 ml) and extracted by ethyl acetate (2×20 ml). Organic layer was separated, washed with water, dried over Na₂SO₄ and evaporated under reduced pressure to get crude product which was further purified by column chromatography (0-5% Ethyl acetate in Hexane) to get the title product. ESI-MS (m/z): 213.05 (M+H)⁺.

### Step 2: (5-methyl-6-(methylthio)pyrimidin-4-yl)methanol

To a stirring solution of product of step 1 (1.70 g, 8.01 mmol) in EtOH (17.00 ml) was added sodium borohydride (0.515 g, 13.61 mmol) portion wise over a period of 10 min. at 25-30° C. The reaction mixture was stirred at 25-30° C. for 1 h. After complete conversion of starting material reaction mixture was cooled to 10° C. and washed with saturated solution of NH₄Cl (15 ml). Organic layer was separated, washed with water, dried over Na₂SO₄ and evaporated under reduced pressure to get the title product. ESI-MS (m/z): 171.60 (M+H)⁺.

### Step 3: 4-(bromomethyl)-5-methyl-6-(methylthio)pyrimidine

To a stirring solution of product of step 2 (1.0 g, 5.87 mmol) in DCM (25 ml) was added TPP (2.0 g, 7.64 mmol) followed by CBr₄ (2.53 g, 7.64 mmol) at 5-10° C. The reaction mixture was stirred at 25-30° C. for 1 h. After complete conversion of starting material reaction mixture further diluted with 15 ml water. Organic layer was separated, washed with water, dried over Na₂SO₄ and evaporated under reduced pressure to get title product which was further purified by column chromatography (0-5% Ethyl acetate in Hexane) to get the title product. ESI-MS (m/z): 234.96 (M+H)⁺.

### Preparation of N-((1-hydroxycyclopropyl)methyl)-6-methyl-1-((5-methyl-6-(methylthio)pyrimidin-4-yl)methyl)-1H-pyrazolo[4,3-b]pyridine-3-carboxamide

### Step 1: ethyl 6-methyl-1-((5-methyl-6-(methylthio)pyrimidin-4-yl)methyl)-1H-pyrazolo[4,3-b]pyridine-3-carboxylate

To a stirring suspension of ethyl 6-methyl-1H-pyrazolo[4,3-b]pyridine-3-carboxylate (0.400 g, 1.94 mmol) and K₂CO₃ (0.404 g, 2.92 mmol) in dry DMF (5 ml) was added Intermediate 4B (0.545 g, 2.33 mmol) at 5-10° C. and the resulting mixture was stirred at 25-30° C. for 1 h. After complete conversion of starting material reaction mixture was diluted with cold water (20 ml) and extracted by ethyl acetate (2×20 ml). Organic layer was separated, washed with water, dried over Na₂SO₄ and evaporated under reduced pressure to get the crude product which was further purified by column chromatography (0-50% Ethyl acetate in Hexane) to get the title product. ESI-MS (m/z): 358.25 (M+H)⁺.

### Step 2: 6-methyl-1-((5-methyl-6-(methylthio)pyrimidin-4-yl)methyl)-1H-pyrazolo[4,3-b]pyridine-3-carboxylic acid

To a stirring solution of product of step 1 (0.55 g, 1.53 mmol) in EtOH (10 ml) was added NaOH (92 mg (2.30 mmol) in 1 ml water). The reaction mixture was stirred at 25-30° C. for 1 h. After complete conversion of starting material reaction mixture was diluted with water (5 ml) and acidified by using dil. HCl. The reaction mixture was extracted with DCM. Organic layer was separated, washed with water, dried over Na₂SO₄ and evaporated under reduced pressure to get the title product. ESI-MS (m/z): 330.14 (M+H)⁺.

### Step 3: N-((1-hydroxycyclopropyl)methyl)-6-methyl-1-((5-methyl-6-(methylthio)pyrimidin-4-yl)methyl)-1H-pyrazolo[4,3-b]pyridine-3-carboxamide

To a stirring solution of product of step 2 (100 mg, 0.304 mmol) in DCM (6 mL) was added EDC.HCl (99 mg, 0.516 mmol) followed by HOBT (70 mg, 0.455 mmol) at 25-30° C. Reaction mixture was stirred for 10 min. at 25-30° C. To this was added 1-(aminomethyl)cyclopropan-1-ol (53 mg, 0.607 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave product as a yellow oil which was further purified by column chromatography (0-60% Ethyl acetate in Hexane) to get title product as solid. ¹H NMR (DMSO-d₆): 8.88-8.87 (m, 1H), 8.59-8.58 (d, J=2.4 Hz, 2H), 8.06 (s, 1H), 5.94 (s, 2H), 5.53 (brs, 1H), 3.54-3.53 (d, J=5.6 Hz, 2H), 2.54 (s, 3H), 2.50 (s, 3H), 2.33 (s, 3H), 0.64-0.56 (m, 4H), ESI-MS (m/z): 399.11 (M+H)⁺.

### Example 5

### Preparation of N-((1-hydroxycyclopropyl)methyl)-1-((6-(2-methoxyethoxy)-5-methylpyrimidin-4-yl)methyl)-6-methyl-1H-pyrazolo[4,3-b]pyridine-3-carboxamide

### Preparation of 4-(chloromethyl)-6-(2-methoxyethoxy)-5-methylpyrimidine (Intermediate 5B)

### Step 1: ethyl 6-(2-methoxyethoxy)-5-methylpyrimidine-4-carboxylate

To a stirring suspension of 60% NaH (0.916 g, 22.93 mmol) in dry THF (10 ml) was added 2-methoxyethanol (1.6 g, 20.93 mmol) drop wise over a period of 15 min. at 0° C. To this was added ethyl 6-chloro-5-methylpyrimidine-4-carboxylate (2 g, 9.97 mmol) portion wise over a period of 5 min. and the resulting mixture was stirred at 0-5° C. for 15 min. After complete conversion of starting material reaction mixture was cooled to 15° C. The reaction mixture was diluted with cold water (25 ml) and extracted by ethyl acetate (2×20 ml). Organic layer was separated, washed with water, dried over Na₂SO₄ and evaporated under reduced pressure to get crude product which was further purified by column chromatography (0-40% Ethyl acetate in Hexane) to get the title product. ESI-MS (m/z): 241.10 (M+H)⁺.

### Step 2: (6-(2-methoxyethoxy)-5-methylpyrimidin-4-yl)methanol

To a stirring solution of product of step 1 (1.0 g, 4.16 mmol) in EtOH (5.00 ml) was added sodium borohydride (0.315 g, 8.32 mmol) portion wise over a period of 10 min. at 25-30° C. The reaction mixture was stirred at 25-30° C. for 1 h. After complete conversion of starting material reaction mixture was cooled to 10° C. and washed with saturated solution of NH₄Cl (15 ml). Organic layer was separated, washed with water, dried over Na₂SO₄ and evaporated under reduced pressure to get the title product. ESI-MS (m/z): 199.14. (M+H)⁺.

### Step 3: 4-(chloromethyl)-6-(2-methoxyethoxy)-5-methylpyrimidine

To a stirring solution of product of step 2 (0.65 g, 3.28 mmol) in DCM (5 ml) was added Thionyl chloride (0.585 g, 4.92 mmol) at 25-30° C. The reaction mixture was stirred at 25-30° C. for 1 h. After complete conversion of starting material solvent was evaporated under vacuum and the residue was diluted with cold water (25 ml) and extracted by DCM (2×10 ml). Organic layer was separated, washed with water, dried over Na₂SO₄ and evaporated under reduced pressure to get title product. ESI-MS (m/z): 217.50 (M+H)⁺.

### Preparation of N-((1-hydroxycyclopropyl)methyl)-1-((6-(2-methoxyethoxy)-5-methylpyrimidin-4-yl)methyl)-6-methyl-1H-pyrazolo[4,3-b]pyridine-3-carboxamide

### Step 1: ethyl 1-((6-(2-methoxyethoxy)-5-methylpyrimidin-4-yl)methyl)-6-methyl-1H-pyrazolo[4,3-b]pyridine-3-carboxylate

To a stirring suspension of ethyl 6-methyl-1H-pyrazolo[4,3-b]pyridine-3-carboxylate (0.200 g, 0.975 mmol) and K₂CO₃ (0.202 g, 1.46 mmol) in dry DMF (5 ml) was added Intermediate 5B (0.275 g, 1.26 mmol) at 25-30° C. Reaction mixture was further stirred at 25-30° C. for 3 h. After complete conversion of starting material reaction mixture was diluted with cold water (20 ml) and extracted by ethyl acetate (2×20 ml). Organic layer was separated, washed with water, dried over Na₂SO₄ and evaporated under reduced pressure to get the crude product which was further purified by column chromatography (0-50% Ethyl acetate in Hexane) to get the title product. ESI-MS (m/z): 386.15 (M+H)⁺.

### Step 2: 1-((6-(2-methoxyethoxy)-5-methylpyrimidin-4-yl)methyl)-6-methyl-1H-pyrazolo[4,3-b]pyridine-3-carboxylic acid

To a stirring solution of product of step 1 (0.150 g, 0.389 mmol) in EtOH (3 ml) was added NaOH (23 mg (0.584 mmol) in 0.5 ml water). The reaction mixture was stirred at 25-30° C. for 1 h. After complete conversion of starting material reaction mixture was diluted with water (10 ml) and acidified by using dil. HCl. The reaction mixture was extracted by DCM (4×30 ml). Organic layer was separated, washed with water, dried over Na₂SO₄ and evaporated under reduced pressure to get the title product. ESI-MS (m/z): 358.11 (M+H)⁺.

### Step 3: N-((1-hydroxycyclopropyl)methyl)-1-((6-(2-methoxyethoxy)-5-methylpyrimidin-4-yl)methyl)-6-methyl-1H-pyrazolo[4,3-b]pyridine-3-carboxamide

To a stirring solution of product of step 2 (120 mg, 0.336 mmol) in DCM (6 mL) was added EDC.HCl (129 mg, 0.672 mmol) followed by HOBT (87 mg, 0.571 mmol) at 25-30° C. Reaction mixture was stirred for 10 min. at 25-30° C. To this was added 1-(aminomethyl)cyclopropan-1-ol (44 mg, 0.504 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave crude product as a yellow oil which was further purified by column chromatography (0-60% Ethyl acetate in Hexane) to get title product as solid. ¹H NMR (DMSO-d₆): 8.88-8.85 (m, 1H), 8.59-8.58 (d, J=1.6 Hz, 1H), 8.37 (s, 1H), 8.07 (s, 1H), 5.90 (s, 2H), 5.53 (s, 1H), 4.49-4.46 (m, 2H), 3.70-3.67 (m, 2H), 3.54-3.52 (d, J=6.0 Hz, 2H), 3.34 (s, 3H), 2.53 (s, 3H), 2.29 (s, 3H), 1.24 (m, 3H), 0.61-0.56 (m, 4H). ESI-MS (m/z): 427.16 (M+H)⁺.

### Example 6

### Preparation of 1-((6-(dimethylamino)-5-methylpyrimidin-4-yl)methyl)-N-((1-hydroxycyclopropyl)methyl)-6-methyl-1H-pyrazolo[4,3-b]pyridine-3-carboxamide

### Step 1: (6-chloro-5-methylpyrimidin-4-yl)methanol

To a stirring solution of ethyl 6-chloro-5-methylpyrimidine-4-carboxylate (3.0 g, 14.95 mmol) in CH₃OH (15 ml) was added sodium borohydride (1.13 g, 29.9 mmol) portion wise over period of 10 min. at 25-30° C. The reaction mixture was stirred at 25-30° C. for 3 h. After complete conversion of starting material reaction mixture was cooled to 10° C. and the reaction mixture was diluted with cold water (100 ml) followed by extraction with ethyl acetate (2×50 ml). Organic layer was separated, washed with water, dried over Na₂SO₄ and evaporated under reduced pressure to get crude product which was used without further purification. ESI-MS (m/z): 159.38 (M+H)⁺.

### Step 2: 4-chloro-6-(chloromethyl)-5-methylpyrimidine

To a stirring solution of product of step 1 (2.2 g, 13.87 mmol) in DCM (22 ml) was added Thionyl chloride (1.50 ml, 20.81 mmol) at 5-10° C. The reaction mixture was stirred at 25-30° C. for 1 h. After complete conversion of starting material, reaction mixture was washed with saturated solution of NaHCO₃, dried over Na₂SO₄ and filtered. Removal of the solvent under reduced pressure gave product as oil. ESI-MS (m/z): 178.34 (M+H)⁺.

### Step 3: ethyl 1-((6-chloro-5-methylpyrimidin-4-yl)methyl)-6-methyl-1H-pyrazolo[4,3-b]pyridine-3-carboxylate

To a stirring suspension of ethyl 6-methyl-1H-pyrazolo[4,3-b]pyridine-3-carboxylate (1.2 g, 5.85 mmol) and K₂CO₃ (1.20 g, 8.77 mmol) in dry DMF (12 ml) was added 4-chloro-6-(chloromethyl)-5-methylpyrimidine (1.24 g, 7.02 mmol) at 5-10° C. Resulting mixture was stirred at 25-30° C. for 3 h. After complete conversion of starting material reaction mixture was diluted with water (20 ml) and extracted by ethyl acetate (2×50 ml). Organic layer was separated, washed with water, dried over Na₂SO₄ and evaporated under reduced pressure to get the crude product which was further purified by column chromatography (0-20% Ethyl acetate in Hexane) to get the title product. ESI-MS (m/z): 346.15. (M+H)*.

### Step 4: ethyl 1-((6-(dimethylamino)-5-methylpyrimidin-4-yl)methyl)-6-methyl-1H-pyrazolo[4,3-b]pyridine-3-carboxylate

To a stirring suspension of product of step 3 (0.350 g, 1.01 mmol) and K₂CO₃ (0.20 g, 1.51 mmol) in dry DMF (5 ml) was added Dimethylamine hydrochloride (0.413 g, 5.06 mmol) at 5-10° C. and the resulting mixture was stirred at 50° C. for 1 h. After complete conversion of starting material reaction mixture was diluted with water (20 ml) and extracted by ethyl acetate (2×20 ml). Organic layer was separated, washed with water, dried over Na₂SO₄ and evaporated under reduced pressure to get the crude product which was further purified by column chromatography (0-20% Ethyl acetate in Hexane) to get the title product. ESI-MS (m/z): 355.33 (M+H)⁺.

### Step 5: 1-((6-(dimethylamino)-5-methylpyrimidin-4-yl)methyl)-6-methyl-1H-pyrazolo[4,3-b]pyridine-3-carboxylic acid

To a stirring solution of product of step 4 (0.26 g, 0.73 mmol) in EtOH (3 ml) was added NaOH (44 mg (1.10 mmol) in 0.5 ml water). The reaction mixture was stirred at 25-30° C. for 1 h. After complete conversion of starting material reaction mixture was diluted with water (10 ml) and the pH was adjusted to 4 by using 5% HCl solution. The reaction mixture was extracted by DCM (3×30 ml). Organic layer was separated, washed with water, dried over Na₂SO₄ and evaporated under reduced pressure to get the title product. ESI-MS (m/z): 327.10 (M+H)⁺.

### Step 6: 1-((6-(dimethylamino)-5-methylpyrimidin-4-yl)methyl)-N-((1-hydroxycyclopropyl)methyl)-6-methyl-1H-pyrazolo[4,3-b]pyridine-3-carboxamide

To a stirring solution of product of step 5 (100 mg, 0.306 mmol) in DCM (3 mL) was added EDC.HCl (117 mg, 0.613 mmol) followed by HOBT (80 mg, 0.521 mmol) and DIPEA (79 mg, 0.613 mmol) at 25-30° C. Reaction mixture was stirred for 10 min. at 25-30° C. To this was added 1-(aminomethyl)cyclopropanol (40 mg, 0.460 mmol) and stirred for 16 h. After 16 h, reaction mixture was diluted with DCM (10 ml). Reaction mixture was washed with saturated solution of NaHCO₃, dried over Na₂SO₄, and filtered. Removal of the solvent under reduced pressure to get the product as a yellow solid, which was further purified by column chromatography (3-5% CH₃OH in Ethyl acetate) to get the title product. ¹H NMR (DMSO-d₆): 8.87-8.85 (m, 1H), 8.58 (s, 1H), 8.19 (s, 1H), 8.06 (s, 1H), 5.79 (s, 2H), 5.53 (brs, 1H), 3.54 (d, J=6 Hz, 2H), 2.50 (s, 3H), 2.96 (s, 6H), 2.30 (s, 3H), 0.61-0.56 (m, 4H). ESI-MS (m/z): 396.19 (M+H)⁺.

### Example 7

### Preparation of N-((1-hydroxycyclopropyl)methyl)-6-methyl-1-((5-methyl-6-morpholinopyrimidin-4-yl)methyl)-1H-pyrazolo[4,3-b]pyridine-3-carboxamide

### Step 1: ethyl 6-methyl-1-((5-methyl-6-morpholinopyrimidin-4-yl)methyl)-1H-pyrazolo[4,3-b]pyridine-3-carboxylate

To a stirring solution of ethyl 1-((6-chloro-5-methylpyrimidin-4-yl)methyl)-6-methyl-1H-pyrazolo[4,3-b]pyridine-3-carboxylate (0.350 g, 1.01 mmol) and K₂CO₃ (0.21 g, 1.51 mmol) in dry DMF (5 ml) was added Morpholine (0.176 g, 2.02 mmol) at 5-10° C. and the resulting mixture was stirred at 50° C. for 3 h. After complete conversion of starting material reaction mixture was diluted with water (20 ml) and extracted by ethyl acetate (3×20 ml). Organic layer was separated, washed with water, dried over Na₂SO₄ and evaporated under reduced pressure to get the crude product which was further purified by column chromatography (0-40% Ethyl acetate in Hexane) to get the title product. ESI-MS (m/z): 397.21 (M+H)⁺.

### Step 2: 6-methyl-1-((5-methyl-6-morpholinopyrimidin-4-yl)methyl)-1H-pyrazolo[4,3-b]pyridine-3-carboxylic acid

To a stirring solution of product of step 1 (0.275 g, 0.69 mmol) in EtOH (3 ml) was added NaOH (42 mg (1.10 mmol) in 0.5 ml water). The reaction mixture was stirred at 25-30° C. for 1 h. After complete conversion of starting material reaction mixture was diluted with water (10 ml) and pH was adjusted to 4 by using 5% HCl solution. The reaction mixture was extracted by DCM (4×30 ml). Organic layer was separated, washed with water, dried over Na₂SO₄ and evaporated under reduced pressure to get the title product. ESI-MS (m/z): 369.51 (M+H)⁺.

### Step 3: N-((1-hydroxycyclopropyl)methyl)-6-methyl-1-((5-methyl-6-morpholinopyrimidin-4-yl)methyl)-1H-pyrazolo[4,3-b]pyridine-3-carboxamide

To a stirring solution of product of step 2 (100 mg, 0.271 mmol) in DCM (3 mL) was added EDC.HCl (104 mg, 0.543 mmol) followed by HOBT (71 mg, 0.461 mmol) and DIPEA (70 mg, 0.543 mmol) at 25-30° C. Reaction mixture was stirred for 10 min. at 25-30° C. To this was added 1-(aminomethyl)cyclopropanol (35 mg, 0.407 mmol) and stirred for 16 h. After 16 h, reaction mixture was diluted with DCM (10 ml). Reaction mixture was washed with saturated solution of NaHCO₃, dried over Na₂SO₄, and filtered. Removal of the solvent under reduced pressure gave product as a yellow solid, which was further purified by column chromatography (3-5% CH₃OH in Ethyl acetate) to get the title product. ¹H NMR (DMSO-d₆): 8.88-8.85 (m, 1H), 8.589-8.585 (d, J=1.6 Hz, 1H), 8.33 (s, 1H), 8.06 (s, 1H), 5.84 (s, 2H), 5.53 (brs, 1H), 3.73-3.71 (t, J=4.0 Hz, 4H), 3.54-3.53 (d, J=5.6 Hz, 2H), 3.30-3.27 (t, J=4.8 Hz, 4H), 2.54 (s, 3H), 2.29 (s, 3H), 0.63-0.56 (m, 4H). ESI-MS (m/z): 438.21 (M+H)⁺.

### Example 8

### Preparation of 1-((6-(cyclopropylmethoxy)-5-methylpyrimidin-4-yl)methyl)-N-((1-hydroxycyclopropyl)methyl)-6-methyl-1H-pyrazolo[4,3-b]pyridine-3-carboxamide

### Step 1: ethyl 6-(cyclopropylmethoxy)-5-methylpyrimidine-4-carboxylate

To a stirring suspension of 60% NaH (0.299 g, 7.48 mmol) in dry THF (10 ml) was added cyclopropylmethanol (0.359 g, 4.98 mmol) drop wise over a period of 15 min. at 0° C. To this was added ethyl 6-chloro-5-methylpyrimidine-4-carboxylate (1 g, 4.98 mmol) portion wise over a period of 5 min. and the resulting mixture was stirred at 0-5° C. for further 15 min. After complete conversion of starting material reaction mixture was diluted with cold water (15 ml) and extracted by ethyl acetate (2×15 ml). Organic layer was separated, washed with water, dried over Na₂SO₄ and evaporated under reduced pressure to get crude product which was further purified by column chromatography (0-30% Ethyl acetate in Hexane) to get the title product. ESI-MS (m/z): 237.10 (M+H)⁺.

### Step 2: (6-(cyclopropylmethoxy)-5-methylpyrimidin-4-yl)methanol

To a stirring solution of product of step 1 (550 mg, 2.328 mmol) in EtOH (17 ml) was added sodium borohydride (106 mg, 2.79 mmol) portion wise over a period of 10 min. at 25-30° C. The reaction mixture was stirred at 25-30° C. for 1 h. After complete conversion of starting material reaction mixture was cooled to 10° C. and washed with saturated solution of NH₄Cl (15 ml). Organic layer was separated, washed with water, dried over Na₂SO₄ and evaporated under reduced pressure to get the title product. ESI-MS (m/z): 195.10 (M+H)⁺.

### Step 3: 4-(bromomethyl)-6-(cyclopropylmethoxy)-5-methylpyrimidine

To a stirring solution of product of step 2 (350 mg, 1.802 mmol) in DCM (10 ml) was added TPP (614 mg, 2.343 mmol) and CBr₄ (777 mg, 2.343 mmol) at 5-10° C. The reaction mixture was stirred at 25-30° C. for 1 h. After complete conversion of starting material, reaction mixture diluted with 15 ml water. Organic layer was separated, washed with water, dried over Na₂SO₄ and evaporated under reduced pressure to get title product which was further purified by column chromatography (0-5% Ethyl acetate in Hexane) to get the title product. ESI-MS (m/z): 259.01 (M+H)⁺.

### Step 4: ethyl 1-((6-(cyclopropylmethoxy)-5-methylpyrimidin-4-yl)methyl)-6-methyl-1H-pyrazolo[4,3-b]pyridine-3-carboxylate

To a stirring suspension of ethyl 6-methyl-1H-pyrazolo[4,3-b]pyridine-3-carboxylate (160 mg, 0.780 mmol) and K₂CO₃ (162 mg, 1.169 mmol) in dry DMF (5 ml) was added product of step 3 (241 mg, 0.936 mmol) at 5-10° C. and the resulting mixture was stirred at 25-30° C. for 1 h. After complete conversion of starting material reaction mixture was diluted with cold water (10 ml) and extracted by ethyl acetate (2×10 ml). Organic layer was separated, washed with water, dried over Na₂SO₄ and evaporated under reduced pressure to get the crude product which was further purified by column chromatography (0-50% Ethyl acetate in Hexane) to get the title product. ESI-MS (m/z): 382.17 (M+H)⁺.

### Step 5: 1-((6-(cyclopropylmethoxy)-5-methylpyrimidin-4-yl)methyl)-6-methyl-1H-pyrazolo[4,3-b]pyridine-3-carboxylic acid

To a stirring solution of product of step 4 (220 mg, 0.577 mmol) in EtOH (10 ml) was added NaOH (34.6 mg (0.865 mmol) in 1 ml water). The reaction mixture was stirred at 25-30° C. for 1 h. After complete conversion of starting material reaction mixture was diluted with water (5 ml) and acidified by using dil. HCl. The reaction mixture was extracted by DCM (3×10 ml). Organic layer was separated, washed with water, dried over Na₂SO₄ and evaporated under reduced pressure to get the title product. ESI-MS (m/z): 354.15 (M+H)⁺.

### Step 6: 1-((6-(cyclopropylmethoxy)-5-methylpyrimidin-4-yl)methyl)-N-((1-hydroxycyclopropyl)methyl)-6-methyl-1H-pyrazolo[4,3-b]pyridine-3-carboxamide

To a stirring solution of product of step 5 (170 mg, 0.481 mmol) in DCM (6 mL) was added EDC.HCl (157 mg, 0.818 mmol) followed by HOBT (111 mg, 0.722 mmol) and DIPEA (0.252 ml, 1.443 mmol) at 25-30° C. Reaction mixture was stirred for 10 min. at 25-30° C. To this was added 1-(aminomethyl)cyclopropan-1-ol (84 mg, 0.962 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave crude product which was further purified by column chromatography (0-60% Ethyl acetate in Hexane) to get title product as solid. ¹H NMR (CDCl₃): 9.07 (bs, 1H), 8.52-8.49 (m, 2H), 7.80 (d, J=0.8 Hz, 1H), 5.71 (s, 2H), 4.21 (d, 2H), 3.75 (d, 2H), 2.54 (s, 3H), 2.32 (s, 3H), 1.28-1.22 (m, 1H), 0.77-0.70 (m, 2H), 0.66-0.63 (m, 2H), 0.61-0.59 (m, 2H), 0.38-0.34 (m, 2H). ESI-MS (m/z): 423.17 (M+H)⁺.

### Example 9

### Preparation of N-((1-hydroxycyclopropyl)methyl)-1-((2-methoxy-6-methylpyridin-3-yl)methyl)-6-methyl-1H-pyrazolo[4,3-b]pyridine-3-carboxamide

### Step 1: 6-methyl-2-oxo-1,2-dihydropyridine-3-carbonitrile

To a stirring suspension of NaOEt (5.97 g, 88 mmol) in diethyl ether (95 ml) was added a solution of ethyl formate (6.44 ml) in acetone (5.85 ml, 80 mmol) at 0° C. The reaction mixture was stirred at 30-40° C. for 2 h. Diethyl ether was evaporated at 50° C. To this was added 2-cyanoacetamide (6.70 g, 80 mmol) dissolved in water (40 ml) followed by piperidine acetate (2.011 g, 13.95 mmol). Reaction mixture was heated to 100° C. for 2 h. After complete conversion of starting material, reaction mixture was cooled to 0° C. and acetic acid was added till pH=5 to get solid product. The reaction mixture was filtered to get the title product. ESI-MS (m/z): 135.12 (M+H)⁺.

### Step 2: 6-methyl-2-oxo-1,2-dihydropyridine-3-carboxylic acid

Product of step 1 (7 g, 52.2 mmol) and 20% aq. NaOH (26 mL) was heated at 140-145° C. for 16 h. The reaction mixture was cooled and pH was adjusted to 8 with 35% HCl and extracted with DCM. The aqueous phase was acidified to get yellow solid which was filtered, washed with water, and dried to get the title compound. ESI-MS (m/z): 154.05 (M+H)⁺.

### Step 3: methyl 2-methoxy-6-methylnicotinate

To a stirring solution of product of step 2 (2.50 g, 16.33 mmol) in CHCl₃ (80 ml) was added silver carbonate (4.50 g, 16.33 mmol) followed by iodomethane (3.06 ml, 49.0 mmol) and reaction mixture was stirred at 25-30° C. for 16 h. After complete conversion of starting material, reaction mixture was diluted with CHCl₃ and water. Organic layer was separated, dried and evaporated to get crude product which was purified by column chromatography to get the title product. ESI-MS (m/z): 182.11 (M+H)⁺.

### Step 4: (2-methoxy-6-methylpyridin-3-yl)methanol

To a stirring solution of methyl product of step 3 (1.75 g, 9.66 mmol) in THF (15 ml) was added LiAlH₄ (0.403 g, 10.62 mmol) at 5-10° C. and stirred for 1 h. After complete conversion of starting material, reaction mixture was quenched with 2 ml water. Decant the organic layer, wash with water and evaporate to get the title product. ESI-MS (m/z): 154.11 (M+H)⁺.

### Step 5: 3-(bromomethyl)-2-methoxy-6-methylpyridine

To a stirring solution of product of step 4 (1.60 g, 10.45 mmol) in DCM (25 ml) was added TPP (4.11 g, 15.67 mmol) and CBr₄ (5.02 g, 15.15 mmol) at 5-10° C. and stirred at 25-30° C. for 3 h. After complete conversion of starting material, reaction mixture was diluted with DCM (20 ml) and water (10 ml). Organic layer was washed with water, dried and evaporated to get crude product which was purified by column chromatography to get the title product. ESI-MS (m/z): 217.98 (M+H)⁺.

### Step 6: ethyl 1-((2-methoxy-6-methylpyridin-3-yl)methyl)-6-methyl-1H-pyrazolo[4,3-b]pyridine-3-carboxylate

To a stirring solution of product of step 5 (0.3 g, 1.462 mmol) in DMF (10 ml) was added K₂CO₃ (0.303 g, 2.193 mmol) and Intermediate 1A (0.379 g, 1.754 mmol) at 25-30° C. and stirred for 3 h. After complete conversion of starting material, reaction mixture was diluted with ethyl acetate (50 ml) and water (10 ml). Organic layer was washed with water, dried and evaporated to get crude product which was purified by column chromatography to get the title product. ESI-MS (m/z): 341.30 (M+H)⁺.

### Step 7: 1-((2-methoxy-6-methylpyridin-3-yl)methyl)-6-methyl-1H-pyrazolo[4,3-b]pyridine-3-carboxylic acid

To a stirring solution of product of step 6 (0.440 g, 1.293 mmol) in EtOH (8.80 ml) and water (1.76 ml) was added NaOH (0.078 g, 1.939 mmol) at 25-30° C. and stirred for 3 h. Reaction mixture was diluted with water (10 ml). Acidification up to pH=4-5 by 35% HCl gave solid product which was filtered to get the title product. ESI-MS (m/z): 313.30 (M+H)⁺.

### Step 8: N-((1-hydroxycyclopropyl)methyl)-1-((2-methoxy-6-methylpyridin-3-yl)methyl)-6-methyl-1H-pyrazolo[4,3-b]pyridine-3-carboxamide

To a stirring solution of product of step 7 (120 mg, 0.384 mmol) in DCM (6 mL) was added EDC.HCl (125 mg, 0.653 mmol) and HOBT (88 mg, 0.576 mmol) at 25-30° C.

Reaction mixture was stirred for 10 min. at 25-30° C. To this was added 1-(aminomethyl)cyclopropan-1-ol (50 mg, 0.576 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave product as a yellow oil which was further purified by column chromatography (0-60% Ethyl acetate in Hexane) to get title product as solid. ¹H NMR (DMSO-d₆): 8.87-8.84 (m, 1H), 8.593-8.590 (d, J=1.2 Hz, 1H), 8.15 (s, 1H), 7.25-7.23 (d, J=7.2 Hz, 1H), 7.80-7.79 (d, J=7.6 Hz, 1H), 5.60 (s, 2H), 5.50 (brs, 1H), 3.82 (s, 3H), 3.53-3.51 (d, J=5.6 Hz, 2H), 2.51 (s, 3H), 2.36 (s, 3H), 0.60-0.55 (m, 4H), ESI-MS (m/z): 382.14 (M+H)⁺.

### Example 10

### Preparation of 1-((6-methoxy-5-methylpyrimidin-4-yl)methyl)-N-(2-methoxyethyl)-6-methyl-1H-pyrazolo[4,3-b]pyridine-3-carboxamide

To a stirring solution of 1-((6-methoxy-5-methylpyrimidin-4-yl)methyl)-6-methyl-1H-pyrazolo[4,3-b]pyridine-3-carboxylic acid (100 mg, 0.319 mmol) in DCM (3 mL) was added EDC.HCl (122 mg, 0.638 mmol) and HOBT (98 mg, 0.638 mmol) at 25-30° C. Reaction mixture was stirred for 10 minutes at 25-30° C. To this was added 2-methoxyethanamine (71 mg, 0.638 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave product as a yellow oil which was further purified by column chromatography (0-60% Ethyl acetate in Hexane) to get title product as solid. ¹H NMR (DMSO-d₆): 8.76-8.73 (t, J=5.2 Hz, 1H), 8.575-8.571 (d, J=1.6 Hz, 1H), 8.39 (s, 1H), 8.06 (s, 1H), 5.89 (s, 2H), 3.94 (s, 3H), 3.61-3.57 (m, 2H), 3.52-3.49 (m, 2H), 3.33 (s, 3H), 2.51 (s, 3H), 2.23 (s, 3H). ESI-MS (m/z): 371.20 (M+H)⁺.

### Example 11

### Preparation of N-((1-hydroxycyclopropyl)methyl)-5-((6-methoxy-5-methylpyrimidin-4-yl)methyl)-3-methyl-5H-pyrrolo[2,3-b]pyrazine-7-carboxamide

### Step 1: 6-chloro-3-((trimethylsilyl)ethynyl)pyrazin-2-amine

To a stirring solution of 3-bromo-6-chloropyrazin-2-amine (10 g, 48.0 mmol) in 67 mL of triethyl amine was added Copper(I) iodide (0.822 g, 4.32 mmol) followed by Palladium(II)bis(triphenylphosphine) (1.684 g, 2.399 mmol) and ethynyltrimethylsilane (5.18 g, 52.8 mmol). The reaction mixture was stirred for 12 h at 25-30° C. After completion of reaction it was diluted with ethyl acetate (100 ml) and 100 ml of water. Organic layer was separated, dried over Na₂SO₄ and evaporated to get desired product. ESI-MS (m/z): 226.09 (M+H)⁺.

### Step 2: 3-chloro-5H-pyrrolo[2,3-b]pyrazine

To a stirring solution of product of step 1 (6 g, 26.6 mmol) in NMP (50 ml) was added Potassium tert-butoxide (5.96 g, 53.2 mmol) and reaction mixture was refluxed for 12 h. After completion of reaction it was diluted with ethyl acetate (100 ml) and 100 ml of water. Organic layer was separated, dried over Na₂SO₄ and evaporated to get oily product. ESI-MS (m/z): 154.04 (M+H)⁺.

### Step 3: 3-chloro-5H-pyrrolo[2,3-b]pyrazine-7-carbaldehyde

To a stirring solution of product of step 2 (20 mg, 0.130 mmol) in acetic acid (1 ml) and water (2.0 ml) was added Hexamethylenetetramine (20.08 mg, 0.143 mmol) and the reaction mixture was heated for 5 h at 120° C. After completion of reaction it was diluted with ethyl acetate (10 ml) and 10 ml of water. Organic layer was separated, dried over Na₂SO₄ and evaporated to get desired product. ESI-MS (m/z): 180.00 (M−H).

### Step 4: 3-chloro-5-((6-methoxy-5-methylpyrimidin-4-yl)methyl)-5H-pyrrolo[2,3-b]pyrazine-7-carbaldehyde

To a stirring solution of 4-(chloromethyl)-6-methoxy-5-methylpyrimidine (47.5 mg, 0.275 mmol) and product of step 3 (50 mg, 0.275 mmol) in DMF (5 ml) was added K₂CO₃ (114 mg, 0.826 mmol). Reaction mixture was stirred for 12 h at 25-30° C. After completion of reaction it was diluted with ethyl acetate (10 ml) and 10 ml of water. Organic layer was separated, dried over Na₂SO₄ evaporated to get desired product. ESI-MS (m/z): 318.07 (M+H)⁺.

### Step 5: 5-((6-methoxy-5-methylpyrimidin-4-yl)methyl)-3-methyl-5H-pyrrolo[2,3-b]pyrazine-7-carbaldehyde

To a stirring solution of product of step 4 (50 mg, 0.157 mmol) in 2 mL Dioxane was added Pd(dppf)C₂ (5.76 mg, 7.87 μmol) followed by K₂CO₃ (65.2 mg, 0.472 mmol) and Methyl boronic acid (11.30 mg, 0.189 mmol). The reaction mixture was stirred for 12 h at 90° C. After completion of reaction it was diluted with ethyl acetate (10 ml) and 10 ml of water. Organic layer was separated, dried over Na₂SO₄ and evaporated to get desired product. ESI-MS (m/z): 298.30 (M+H)⁺.

### Step 6: 5-((6-methoxy-5-methylpyrimidin-4-yl)methyl)-3-methyl-5H-pyrrolo[2,3-b]pyrazine-7-carboxylic acid

To a stirring solution of product of step 5 (20 mg, 0.067 mmol) in tert-butanol (2 ml) was added a solution of sodium chlorite (30.4 mg, 0.336 mmol) and sodium phosphate mono basic (38.2 mg, 0.269 mmol) dissolved in 1 mL of water. Reaction mixture and stirred for 3 h at 25-30° C. After completion of reaction it was diluted with ethyl acetate (10 ml) and 10 ml of water. Organic layer was separated, dried over Na₂SO₄ and evaporated to get desired product. ESI-MS (m/z): 314.20 (M+H)⁺.

### Step 7: N-((1-hydroxycyclopropyl)methyl)-5-((6-methoxy-5-methylpyrimidin-4-yl)methyl)-3-methyl-5H-pyrrolo[2,3-b]pyrazine-7-carboxamide

To a stirring solution of product of step 6 (100 mg, 0.319 mmol) in DCM (5.0 ml) was added EDC:HCl (86 mg, 0.451 mmol) followed by HOBT (58.8 mg, 0.372 mmol). To this was added 1-(aminomethyl)cyclopropan-1-ol (28.2 mg, 0.312 mmol). The reaction mixture was stirred at 25-30° C. for 16 h. After completion of reaction it was diluted with ethyl acetate (10 ml) and 10 ml of water. Organic layer was separated, dried over Na₂SO₄ and evaporated to get desired product. ¹H NMR (DMSO-d₆): 8.51 (s, 1H), 8.40 (s, 1H), 8.37-8.35 (d, J=8.4 Hz, 2H), 5.76 (s, 2H), 5.50 (s, 1H), 3.94 (s, 3H), 3.51-3.32 (m, 2H), 2.67 (s, 3H), 2.33 (s, 3H), 0.63-0.55 (m, 2H), 3.57-3.55 (m, 2H). ESI-MS (m/z): 383.18 (M+H)⁺.

### Example 12

### Preparation of N-((1-hydroxycyclopropyl)methyl)-3-methoxy-5-((6-methoxy-5-methylpyrimidin-4-yl)methyl)-5H-pyrrolo[2,3-b]pyrazine-7-carboxamide

### Step 1: 3-methoxy-5-((6-methoxy-5-methylpyrimidin-4-yl)methyl)-5H-pyrrolo[2,3-b]pyrazine-7-carbaldehyde

To a stirring solution of 3-chloro-5-((6-methoxy-5-methylpyrimidin-4-yl)methyl)-5H-pyrrolo[2,3-b]pyrazine-7-carbaldehyde (10 mg, 0.0.031 mmol) in CH₃OH (1 ml) was added NaOCH₃ (2.55 mg, 0.047 mmol). Reaction mixture was stirred for 5 h at 25-30° C. After completion of reaction it was diluted with ethyl acetate (10 ml) and 10 ml of water. Organic layer was separated, dried over Na₂SO₄ and evaporated to get desired product. ESI-MS (m/z): 314.10 (M+H)⁺.

### Step 2: 3-methoxy-5-((6-methoxy-5-methylpyrimidin-4-yl)methyl)-5H-pyrrolo[2,3-b]pyrazine-7-carboxylic acid

To a stirring solution of product of step 1 (20 mg, 0.067 mmol) in tert-butanol (2 ml) was added a solution of sodium chlorite (30.4 mg, 0.336 mmol) and sodium phosphate monobasic (38.2 mg, 0.269 mmol) dissolved in 1 mL of water and reaction mixture was stirred for 3 h at 25-30° C. After completion of reaction it was diluted with ethyl acetate (10 ml) and 10 ml of water. Organic layer was separated, dried over Na₂SO₄ and evaporated to get desired product. ESI-MS (m/z): 330.31 (M+H)⁺.

### Step 3: N-((1-hydroxycyclopropyl)methyl)-3-methoxy-5-((6-methoxy-5-methylpyrimidin-4-yl)methyl)-5H-pyrrolo[2,3-b]pyrazine-7-carboxamide

To a stirring solution of product of step 2 (100 mg, 0.319 mmol) in DCM (5.0 ml) was added EDC:HCl (87 mg, 0.459 mmol) and HOBT (55.8 mg, 0.364 mmol). To this was added 1-(aminomethyl)cyclopropan-1-ol (26.5 mg, 0.304 mmol). The reaction mixture was stirred at 25-30° C. for 16 h. After completion of reaction it was diluted with ethyl acetate (10 ml) and 10 ml of water. Organic layer was separated, dried over Na₂SO₄ and evaporated to get desired product. ¹H NMR (DMSO-d₆): 8.46 (s, 1H), 8.26 (s, 2H), 8.17 (s, 1H), 5.58 (s, 2H), 5.48 (s, 1H), 3.94 (s, 3H), 3.89 (s, 3H), 3.49-3.48 (d, J=5.6 Hz, 2H), 2.32 (s, 3H), 1.05-1.03 (t, J=6.4 Hz, 2H), 0.61-0.60 (d, J=3.6 Hz, 2H), 0.57-0.56 (d, J=3.6 Hz, 2H). ESI-MS (m/z): 399.16 (M+H)⁺.

### Example 13

### Preparation of 5-((6-ethoxy-5-methylpyrimidin-4-yl)methyl)-N-((1-hydroxycyclopropyl)methyl)-3-methyl-5H-pyrrolo[2,3-b]pyrazine-7-carboxamide

### Step 1: 3-chloro-5-((6-ethoxy-5-methylpyrimidin-4-yl)methyl)-5H-pyrrolo[2,3-b]pyrazine-7-carbaldehyde

To a stirring solution of 4-(chloromethyl)-6-ethoxy-5-methylpyrimidine (50 mg, 0.281 mmol) and 3-chloro-5H-pyrrolo[2,3-b]pyrazine-7-carbaldehyde (55 mg, 0.281 mmol) in DMF (5 ml) was added K₂CO₃ (122 mg, 0.831 mmol). Reaction mixture was stirred for 12 h at 25-30° C. After completion of reaction it was diluted with ethyl acetate (10 ml) and 10 ml of water. Organic layer was separated, dried over Na₂SO₄ and evaporated to get desired product. ESI-MS (m/z): 332.73 (M+H)⁺.

### Step 2: 5-((6-ethoxy-5-methylpyrimidin-4-yl)methyl)-3-methyl-5H-pyrrolo[2,3-b]pyrazine-7-carbaldehyde

To a stirring solution of product of step 1 (100 mg, 0.32 mmol) in 2 mL Dioxane was added Pd(dppf)Cl₂ (11.46 mg, 15.67 μmol) followed by K₂CO₃ (13.4 mg, 0.94 mmol) and methyl boronic acid (22.60 mg, 0.38 mmol). The reaction mixture was stirred for 12 h at 90° C. After completion of reaction it was diluted with ethyl acetate (10 ml) and 10 ml of water. Organic layer was separated, dried over Na₂SO₄ and evaporated to get desired product. ESI-MS (m/z): 312.20 (M+H)⁺.

### Step 3: 5-((6-ethoxy-5-methylpyrimidin-4-yl)methyl)-3-methyl-5H-pyrrolo[2,3-b]pyrazine-7-carboxylic acid

To a stirring solution of product of step 2 (60 mg, 0.20 mmol) in tert-butanol (2 ml) was added a solution of sodium chlorite (90.9 mg, 0.99 mmol) and sodium phosphate monobasic (114.2 mg, 0.78 mmol) dissolved in 1 mL of water. Reaction mixture was stirred for 3 h at 25-30° C. After completion of reaction it was diluted with ethyl acetate (10 ml) and 10 ml of water. Organic layer was separated, dried over Na₂SO₄ and evaporated to get desired product. ESI-MS (m/z): 328.20 (M+H)⁺.

### Step 4: Preparation of 5-((6-ethoxy-5-methylpyrimidin-4-yl)methyl)-N-((1-hydroxycyclopropyl)methyl)-3-methyl-5H-pyrrolo[2,3-b]pyrazine-7-carboxamide

To a stirring solution of product of step 3 (100 mg, 0.319 mmol) in DCM (5.0 ml) was added EDC:HCl (87 mg, 0.459 mmol) and HOBT (55.8 mg, 0.364 mmol). To this was added 1-(aminomethyl)cyclopropan-1-ol (26.5 mg, 0.304 mmol). The reaction mixture was stirred at 25-30° C. for 16 h. After completion of reaction it was diluted with ethyl acetate (10 ml) and 10 ml of water. Organic layer was separated, dried over Na₂SO₄ and evaporated to get desired product. ¹H NMR (DMSO-d₆): 8.51 (s, 1H), 8.37-8.34 (m, 3H), 5.64 (s, 2H), 5.50 (s, 1H), 4.41-4.39 (q, J=7.2 Hz, 2H), 3.51-3.49 (d, J=8.4 Hz, 2H), 2.28 (s, 3H), 1.36-1.32 (t, J=7.2 Hz, 3H), 0.61-0.57 (m, 4H). ESI-MS (m/z): 397.18 (M+H)⁺.

### Example 14

### Preparation of N-((1-hydroxycyclopropyl)methyl)-1-((6-methoxy-5-methylpyrimidin-4-yl)methyl)-6-methyl-1H-pyrazolo[3,4-b]pyrazine-3-carboxamide

### Step 1: ethyl 2-(3,5-dichloropyrazin-2-yl)-2-oxoacetate

To a stirring solution of 2,6-dichloropyrazine (2 g, 13.43 mmol) and diethyl oxalate (2.158 g, 14.77 mmol) in dry THF (20 ml) was added Lithium 2,2,6,6-tetramethylpiperidide (593 mg, 4.03 mmol) at −60° C. over a period of 15 min. and the resulting mixture was stirred at −30° C. for 6 h. After complete conversion of starting material reaction mixture was diluted with water (50 ml) and extracted by ethyl acetate (2×50 ml). Organic layer was separated, washed with water, dried over Na₂SO₄ and evaporated under reduced pressure to get crude product which was further purified by column chromatography (0-40% Ethyl acetate in Hexane) to get the title product. ESI-MS (m/z): 250.04 (M+H)⁺.

### Step 2: ethyl-2-(3,5-dichloropyrazin-2-yl)-2-hydrazineylideneacetate

To a stirring solution of product of step 1 (660 mg, 2.65 mmol) in EtOH (10 ml) was added hydrazine hydrobromide (299 mg, 2.65 mmol). The reaction mixture was stirred at 25-30° C. for 10 h. After complete conversion of starting material reaction mixture was cooled to 10° C. and washed with saturated solution of NaHCO₃ (150 ml). Organic layer was separated, washed with water, dried over Na₂SO₄ and evaporated under reduced pressure to get the title product. ESI-MS (m/z): 263.0 (M+H)⁺.

### Step 3: ethyl 6-chloro-1H-pyrazolo[3,4-b]pyrazine-3-carboxylate

To a stirring solution of product of step 2 (1 g, 3.80 mmol) in THF (10 ml) was added sodium hydride (0.27 g, 11.40 mmol) at 0° C. The reaction mixture was stirred at 25-30° C. for 2 h. After complete conversion of starting material reaction mixture was cooled to 10° C. and washed with saturated solution of NaHCO₃ (150 ml). Organic layer was separated, washed with water, dried over Na₂SO₄ and evaporated under reduced pressure to get the title product. ESI-MS (m/z): 225.0 (M−H).

### Step 4: ethyl 6-chloro-1-((6-methoxy-5-methylpyrimidin-4-yl)methyl)-1H-pyrazolo[3,4-b]pyrazine-3-carboxylate

To a stirring solution of product of step 3 (1 g, 4.41 mmol) in DMF (10 ml) was added 4-(chloromethyl)-6-methoxy-5-methylpyrimidine (0.914 g, 5.30 mmol) and K₂CO₃ (0.915 g, 6.62 mmol). The reaction mixture was stirred at 25-30° C. for 12 h. After complete conversion of starting material reaction mixture was added to cold water. The precipitated product was filtered, washed with water, and dried to get the title product. ESI-MS (m/z): 363.05 (M+H)⁺.

### Step 5: ethyl 1-((6-methoxy-5-methylpyrimidin-4-yl)methyl)-6-methyl-1H-pyrazolo[3,4-b]pyrazine-3-carboxylate

To a stirring solution of product of step 4 (10 mg, 0.028 mmol) in Dioxane (2 ml) was added Pd(dppf)Cl₂ (2.017 mg, 2.76 μmol) followed by methyl boronic acid (3.30 mg, 0.055 mmol) and cesium carbonate (13.47 mg, 0.041 mmol). The reaction mixture was stirred at 25-30° C. for 12 h. After complete conversion of starting material reaction mixture was diluted with water. The precipitated product was filtered, washed with water, dried to get the title product. ESI-MS (m/z): 343.30 (M+H)⁺.

### Step 6: 1-((6-methoxy-5-methylpyrimidin-4-yl)methyl)-6-methyl-1H-pyrazolo[3,4-b]pyrazine-3-carboxylic acid

To a stirring solution of product of step 5 (150 mg, 0.438 mmol) in EtOH (4 ml) was added NaOH (52.6 mg (1.314 mmol) in 1 ml water). The reaction mixture was stirred at 25-30° C. for 1 h. After complete conversion of starting material reaction mixture was diluted with water (5 ml) and acidified by using dil. HCl. The reaction mixture was extracted by ethyl acetate (2×10 ml). Organic layer was separated, washed with water, dried over Na₂SO₄ and evaporated under reduced pressure to get the title product. ESI-MS (m/z): 315.20 (M+H)⁺.

### Step 7: N-((1-hydroxycyclopropyl)methyl)-1-((6-methoxy-5-methylpyrimidin-4-yl)methyl)-6-methyl-1H-pyrazolo[3,4-b]pyrazine-3-carboxamide

To a stirring solution of product of step 6 (80 mg, 0.242 mmol) in DCM (5 ml) was added EDC:HCl (65 mg, 0.399 mmol) and HOBT (55.6 mg, 0.363 mmol). To this was added 1-(aminomethyl)cyclopropan-1-ol (31.7 mg, 0.363 mmol). The reaction mixture was stirred at 25-30° C. for 16 h. After completion of reaction it was diluted with ethyl acetate (10 ml) and 10 ml of water. Organic layer was separated, dried over Na₂SO₄ and evaporated to get the title product. ¹H NMR (DMSO-d₆): 8.86 (s, 1H), 8.37-8.38 (m, 2H), 5.90 (s, 2H), 5.53 (s, 1H), 3.95 (s, 3H), 3.52-3.50 (d, J=5.6 Hz, 2H), 2.67 (s, 3H), 2.27 (s, 3H), 1.04-1.03 (d, J=6.0 Hz, 1H), 0.63-0.56 (m, 4H). ESI-MS (m/z): 384.13 (M+H)⁺.

### Example 15

### Preparation of N-((1-hydroxycyclopropyl)methyl)-6-methoxy-1-((6-methoxy-5-methylpyrimidin-4-yl)methyl)-1H-pyrazolo[3,4-b]pyrazine-3-carboxamide

### Step 1: ethyl 6-methoxy-1-((6-methoxy-5-methylpyrimidin-4-yl)methyl)-1H-pyrazolo[3,4-b]pyrazine-3-carboxylate

To a stirring solution of ethyl 6-chloro-1-((6-methoxy-5-methylpyrimidin-4-yl)methyl)-1H-pyrazolo[3,4-b]pyrazine-3-carboxylate (150 mg, 0.413 mmol) in CH₃OH (1 ml) was added NaOCH₃ (22.34 mg, 0.413 mmol) and the reaction mixture was stirred for 5 h at 25-30° C. After completion of reaction it was diluted with ethyl acetate (10 ml) and 10 ml of water. Organic layer was separated, dried over Na₂SO₄ and evaporated to get desired product. ESI-MS (m/z): 359.10 (M+H)⁺.

### Step 2: 6-methoxy-1-((6-methoxy-5-methylpyrimidin-4-yl)methyl)-1H-pyrazolo[3,4-b]pyrazine-3-carboxylic acid

To a stirring solution of product of step 1 (110 mg, 0.307 mmol) in EtOH (4 ml) was added NaOH (36.8 mg (0.92 mmol) in 1 ml water). The reaction mixture was stirred at 25-30° C. for 1 h. After complete conversion of starting material reaction mixture was diluted with water (5 ml) and acidified by using dil. HCl. The reaction mixture was extracted by ethyl acetate (2×10 ml). Organic layer was separated, washed with water, dried over Na₂SO₄ and evaporated under reduced pressure to get the title product. ESI-MS (m/z): 331.10 (M+H)⁺.

### Step 3: Preparation of N-((1-hydroxycyclopropyl)methyl)-6-methoxy-1-((6-methoxy-5-methylpyrimidin-4-yl)methyl)-1H-pyrazolo[3,4-b]pyrazine-3-carboxamide

To a stirring solution of product of step 2 (80 mg, 0.242 mmol) in DCM (5 ml) was added EDC:HCl (65 mg, 0.399 mmol) and HOBT (55.6 mg, 0.363 mmol). To this was added 1-(aminomethyl)cyclopropan-1-ol (31.7 mg, 0.363 mmol). The reaction mixture was stirred at 25-30° C. for 16 h. After completion of reaction it was diluted with ethyl acetate (10 ml) and 10 ml of water. Organic layer was separated, dried over Na₂SO₄ and evaporated to get desired product. ¹H NMR (DMSO-d₆): 8.79-8.43 (m, 3H), 8.28 (s, 11H), 5.77 (s, 2H), 5.52 (s, 11H), 4.48 (m, 2H), 3.98 (s, 3H), 3.48 (s, 3H), 2.67 (s, 3H), 2.26-2.50 (m, 4H), 0.63-0.56 (in, 4H).

Using appropriate starting materials and suitable modifications of the process described in above examples, including suitable addition and/or deletion of steps as may be necessary, well within the scope of a person skilled in the art the following compounds were prepared in an analogues manner.

TABLE 1

| | | ESI-MS |
|---|---|---|
| EX. | | (m/z) |
| No. | Name | (M + H)⁺ |
| 16 | N-((1-hydroxycyclopropyl)methyl)-6-methyl-1-((5-methyl-6-(oxetan-3- | 425.17 |
| | yloxy)pyrimidin-4-yl)methyl)-1H-pyrazolo[4,3-b]pyridine-3-carboxamide | |
| 17 | N-((1-hydroxycyclopropyl)methyl)-6-methoxy-1-((6-methoxy-5- | 398.15 |
| | methylpyrimidin-4-yl)methyl)-1H-pyrazolo[4,3-b]pyridine-3- | |
| | carboxamide | |
| 18 | 1-((6-ethoxy-5-methylpyrimidin-4-yl)methyl)-N-((1- | 413.18 |
| | hydroxycyclopropyl)methyl)-6-methoxy-1H-pyrazolo[4,3-b]pyridine-3- | |
| | carboxamide | |
| 19 | 1-((6-(dimethylamino)-5-methylpyrimidin-4-yl)methyl)-N-((1- | 412.19 |
| | hydroxycyclopropyl)methyl)-6-methoxy-1H-pyrazolo[4,3-b]pyridine-3- | |
| | carboxamide | |
| 20 | 1-((6-(ethylthio)-5-methylpyrimidin-4-yl)methyl)-N-((1- | 413.16 |
| | hydroxycyclopropyl)methyl)-6-methyl-1H-pyrazolo[4,3-b]pyridine-3- | |
| | carboxamide | |
| 21 | 1-((6-(difluoromethoxy)-5-methylpyrimidin-4-yl)methyl)-N-((1- | 419.15 |
| | hydroxycyclopropyl)methyl)-6-methyl-1H-pyrazolo[4,3-b]pyridine-3- | |
| | carboxamide | |
| 22 | N-((1-fluorocyclopropyl)methyl)-1-((6-methoxy-5-methylpyrimidin-4- | 385.16 |
| | yl)methyl)-6-methyl-1H-pyrazolo[4,3-b]pyridine-3-carboxamide | |
| 23 | 1-((5-fluoro-2,6-dimethylpyridin-3-yl)methyl)-N-((1- | 384.17 |
| | hydroxycyclopropyl)methyl)-6-methyl-1H-pyrazolo[4,3-b]pyridine-3- | |
| | carboxamide | |
| 24 | 1-((3,5-dimethylpyrazin-2-yl)methyl)-N-((1- | 367.16 |
| | hydroxycyclopropyl)methyl)-6-methyl-1H-pyrazolo[4,3-b]pyridine-3- | |
| | carboxamide | |
| 25 | 6-chloro-N-((1-hydroxycyclopropyl)methyl)-1-((6-methoxy-5- | 403.11 |
| | methylpyrimidin-4-yl)methyl)-1H-pyrazolo[4,3-b]pyridine-3- | |
| | carboxamide | |
| 26 | 1-((5-fluoro-2-methoxy-6-methylpyridin-3-yl)methyl)-N-((1- | 400.16 |
| | hydroxycyclopropyl)methyl)-6-methyl-1H-pyrazolo[4,3-b]pyridine-3- | |
| | carboxamide | |
| 27 | N-((1-hydroxycyclopropyl)methyl)-1-((2-methoxy-5- | 436.14 |
| | (trifluoromethyl)pyridin-3-yl)methyl)-6-methyl-1H-pyrazolo[4,3- | |
| | b]pyridine-3-carboxamide | |
| 28 | 1-((5-fluoro-2-methoxypyridin-3-yl)methyl)-N-((1- | 386.15 |
| | hydroxycyclopropyl)methyl)-6-methyl-1H-pyrazolo[4,3-b]pyridine-3- | |
| | carboxamide | |
| 29 | 1-(2,4-dimethylbenzyl)-N-((1-hydroxycyclopropyl)methyl)-6-methyl-1H- | 365.18 |
| | pyrazolo[4,3-b]pyridine-3-carboxamide | |
| 30 | 5-((6-ethoxy-5-methylpyrimidin-4-yl)methyl)-N-((1- | 413.18 |
| | hydroxycyclopropyl)methyl)-3-methoxy-5H-pyrrolo[2,3-b]pyrazine-7- | |
| | carboxamide | |
| 31 | 5-((6-(dimethylamino)-5-methylpyrimidin-4-yl)methyl)-N-((1- | 396.20 |
| | hydroxycyclopropyl)methyl)-3-methyl-5H-pyrrolo[2,3-b]pyrazine-7- | |
| | carboxamide | |
| 32 | 5-((6-(dimethylamino)-5-methylpyrimidin-4-yl)methyl)-N-((1- | 412.18 |
| | hydroxycyclopropyl)methyl)-3-methoxy-5H-pyrrolo[2,3-b]pyrazine-7- | |
| | carboxamide | |
| 33 | N-((1-hydroxycyclopropyl)methyl)-3-methoxy-5-((5-methyl-6- | 415.14 |
| | (methylthio)pyrimidin-4-yl)methyl)-5H-pyrrolo[2,3-b]pyrazine-7- | |
| | carboxamide | |
| 34 | N-((1-hydroxycyclopropyl)methyl)-3-methyl-5-((5-methyl-6- | 399.17 |
| | (methylthio)pyrimidin-4-yl)methyl)-5H-pyrrolo[2,3-b]pyrazine-7- | |
| | carboxamide | |
| 35 | 5-((6-(ethylthio)-5-methylpyrimidin-4-yl)methyl)-N-((1- | 413.15 |
| | hydroxycyclopropyl)methyl)-3-methyl-5H-pyrrolo[2,3-b]pyrazine-7- | |
| | carboxamide | |
| 36 | 5-((6-(ethylthio)-5-methylpyrimidin-4-yl)methyl)-N-((1- | 429.17 |
| | hydroxycyclopropyl)methyl)-3-methoxy-5H-pyrrolo[2,3-b]pyrazine-7- | |
| | carboxamide | |
| 37 | 5-((6-(difluoromethoxy)-5-methylpyrimidin-4-yl)methyl)-N-((1- | 419.18 |
| | hydroxycyclopropyl)methyl)-3-methyl-5H-pyrrolo[2,3-b]pyrazine-7- | |
| | carboxamide | |
| 38 | 5-((6-(difluoromethoxy)-5-methylpyrimidin-4-yl)methyl)-N-((1- | 435.13 |
| | hydroxycyclopropyl)methyl)-3-methoxy-5H-pyrrolo[2,3-b]pyrazine-7- | |
| | carboxamide | |
| 39 | N-((1-fluorocyclopropyl)methyl)-5-((6-methoxy-5-methylpyrimidin-4- | 385.15 |
| | yl)methyl)-3-methyl-5H-pyrrolo[2,3-b]pyrazine-7-carboxamide | |
| 40 | N-((1-fluorocyclopropyl)methyl)-3-methoxy-5-((6-methoxy-5- | 401.18 |
| | methylpyrimidin-4-yl)methyl)-5H-pyrrolo[2,3-b]pyrazine-7-carboxamide | |
| 41 | N-((1-hydroxycyclopropyl)methyl)-5-((2-methoxy-6-methylpyridin-3- | 382.16 |
| | yl)methyl)-3-methyl-5H-pyrrolo[2,3-b]pyrazine-7-carboxamide | |
| 42 | N-((1-hydroxycyclopropyl)methyl)-3-methoxy-5-((2-methoxy-6- | 398.16 |
| | methylpyridin-3-yl)methyl)-5H-pyrrolo[2,3-b]pyrazine-7-carboxamide | |
| 43 | 5-((5-fluoro-2,6-dimethylpyridin-3-yl)methyl)-N-((1- | 384.19 |
| | hydroxycyclopropyl)methyl)-3-methyl-5H-pyrrolo[2,3-b]pyrazine-7- | |
| | carboxamide | |
| 44 | 5-((5-fluoro-2,6-dimethylpyridin-3-yl)methyl)-N-((1- | 400.15 |
| | hydroxycyclopropyl)methyl)-3-methoxy-5H-pyrrolo[2,3-b]pyrazine-7- | |
| | carboxamide | |
| 45 | 5-((3,5-dimethylpyrazin-2-yl)methyl)-N-((1- | 367.17 |
| | hydroxycyclopropyl)methyl)-3-methyl-5H-pyrrolo[2,3-b]pyrazine-7- | |
| | carboxamide | |
| 46 | 5-((3,5-dimethylpyrazin-2-yl)methyl)-N-((1- | 383.16 |
| | hydroxycyclopropyl)methyl)-3-methoxy-5H-pyrrolo[2,3-b]pyrazine-7- | |
| | carboxamide | |
| 47 | N-((1-hydroxycyclopropyl)methyl)-5-((6-methoxy-5-methylpyrimidin-4- | 369.10 |
| | yl)methyl)-5H-pyrrolo[2,3-b]pyrazine-7-carboxamide | |
| 48 | 5-((5-fluoro-2-methoxy-6-methylpyridin-3-yl)methyl)-N-((1- | 400.19 |
| | hydroxycyclopropyl)methyl)-3-methyl-5H-pyrrolo[2,3-b]pyrazine-7- | |
| | carboxamide | |
| 49 | 5-((5-fluoro-2-methoxy-6-methylpyridin-3-yl)methyl)-N-((1- | 416.15 |
| | hydroxycyclopropyl)methyl)-3-methoxy-5H-pyrrolo[2,3-b]pyrazine-7- | |
| | carboxamide | |
| 50 | 5-((5-fluoro-2-methoxypyridin-3-yl)methyl)-N-((1- | 386.14 |
| | hydroxycyclopropyl)methyl)-3-methyl-5H-pyrrolo[2,3-b]pyrazine-7- | |
| | carboxamide | |
| 51 | 5-((5-fluoro-2-methoxypyridin-3-yl)methyl)-N-((1- | 402.13 |
| | hydroxycyclopropyl)methyl)-3-methoxy-5H-pyrrolo[2,3-b]pyrazine-7- | |
| | carboxamide | |
| 52 | N-((1-hydroxycyclopropyl)methyl)-5-((2-methoxy-5- | 436.16 |
| | (trifluoromethyl)pyridin-3-yl)methyl)-3-methyl-5H-pyrrolo[2,3- | |
| | b]pyrazine-7-carboxamide | |
| 53 | N-((1-hydroxycyclopropyl)methyl)-3-methoxy-5-((2-methoxy-5- | 452.14 |
| | (trifluoromethyl)pyridin-3-yl)methyl)-5H-pyrrolo[2,3-b]pyrazine-7- | |
| | carboxamide | |
| 54 | 3-(dimethylamino)-N-((1-hydroxycyclopropyl)methyl)-5-((6-methoxy-5- | 412.18 |
| | methylpyrimidin-4-yl)methyl)-5H-pyrrolo[2,3-b]pyrazine-7-carboxamide | |
| 55 | 3-cyano-N-((1-hydroxycyclopropyl)methyl)-5-((6-methoxy-5- | 394.17 |
| | methylpyrimidin-4-yl)methyl)-5H-pyrrolo[2,3-b]pyrazine-7-carboxamide | |
| 56 | 3-ethoxy-N-((1-hydroxycyclopropyl)methyl)-5-((6-methoxy-5- | 413.17 |
| | methylpyrimidin-4-yl)methyl)-5H-pyrrolo[2,3-b]pyrazine-7-carboxamide | |
| 57 | N-((1-hydroxycyclopropyl)methyl)-3-isopropoxy-5-((6-methoxy-5- | 427.18 |
| | methylpyrimidin-4-yl)methyl)-5H-pyrrolo[2,3-b]pyrazine-7-carboxamide | |
| 58 | N-((1-hydroxycyclopropyl)methyl)-6-methyl-1-((5-methyl-6- | 400.12 |
| | (methylthio)pyrimidin-4-yl)methyl)-1H-pyrazolo[3,4-b]pyrazine-3- | |
| | carboxamide | |
| 59 | N-((1-hydroxycyclopropyl)methyl)-6-methoxy-1-((5-methyl-6- | 416.15 |
| | (methylthio)pyrimidin-4-yl)methyl)-1H-pyrazolo[3,4-b]pyrazine-3- | |
| | carboxamide | |
| 60 | 1-((6-(dimethylamino)-5-methylpyrimidin-4-yl)methyl)-N-((1- | 413.18 |
| | hydroxycyclopropyl)methyl)-6-methoxy-1H-pyrazolo[3,4-b]pyrazine-3- | |
| | carboxamide | |
| 61 | 1-((6-(dimethylamino)-5-methylpyrimidin-4-yl)methyl)-N-((1- | 397.18 |
| | hydroxycyclopropyl)methyl)-6-methyl-1H-pyrazolo[3,4-b]pyrazine-3- | |
| | carboxamide | |
| 62 | 1-((6-ethoxy-5-methylpyrimidin-4-yl)methyl)-N-((1- | 398.17 |
| | hydroxycyclopropyl)methyl)-6-methyl-1H-pyrazolo[3,4-b]pyrazine-3- | |
| | carboxamide | |
| 63 | 1-((6-ethoxy-5-methylpyrimidin-4-yl)methyl)-N-((1- | 414.16 |
| | hydroxycyclopropyl)methyl)-6-methoxy-1H-pyrazolo[3,4-b]pyrazine-3- | |
| | carboxamide | |
| 64 | 1-((6-(difluoromethoxy)-5-methylpyrimidin-4-yl)methyl)-N-((1- | 420.13 |
| | hydroxycyclopropyl)methyl)-6-methyl-1H-pyrazolo[3,4-b]pyrazine-3- | |
| | carboxamide | |
| 65 | 1-((6-(difluoromethoxy)-5-methylpyrimidin-4-yl)methyl)-N-((1- | 436.16 |
| | hydroxycyclopropyl)methyl)-6-methoxy-1H-pyrazolo[3,4-b]pyrazine-3- | |
| | carboxamide | |
| 66 | N-((1-hydroxycyclopropyl)methyl)-1-((2-methoxy-6-methylpyridin-3- | 383.16 |
| | yl)methyl)-6-methyl-1H-pyrazolo[3,4-b]pyrazine-3-carboxamide | |
| 67 | N-((1-hydroxycyclopropyl)methyl)-6-methoxy-1-((2-methoxy-6- | 399.15 |
| | methylpyridin-3-yl)methyl)-1H-pyrazolo[3,4-b]pyrazine-3-carboxamide | |
| 68 | 1-((5-fluoro-2,6-dimethylpyridin-3-yl)methyl)-N-((1- | 385.16 |
| | hydroxycyclopropyl)methyl)-6-methyl-1H-pyrazolo[3,4-b]pyrazine-3- | |
| | carboxamide | |
| 69 | 1-((5-fluoro-2,6-dimethylpyridin-3-yl)methyl)-N-((1- | 401.18 |
| | hydroxycyclopropyl)methyl)-6-methoxy-1H-pyrazolo[3,4-b]pyrazine-3- | |
| | carboxamide | |
| 70 | 1-((3,5-dimethylpyrazin-2-yl)methyl)-N-((1-hydroxycyclopropyl) | 368.16 |
| | methyl)-6-methyl-1H-pyrazolo[3,4-b]pyrazine-3-carboxamide | |
| 71 | 1-((3,5-dimethylpyrazin-2-yl)methyl)-N-((1-hydroxycyclopropyl) | 384.15 |
| | methyl)-6-methoxy-1H-pyrazolo[3,4-b]pyrazine-3-carboxamide | |
| 72 | N-((1-fluorocyclopropyl)methyl)-1-((6-methoxy-5-methylpyrimidin-4- | 386.16 |
| | yl)methyl)-6-methyl-1H-pyrazolo[3,4-b]pyrazine-3-carboxamide | |
| 73 | N-((1-fluorocyclopropyl)methyl)-6-methoxy-1-((6-methoxy-5- | 402.14 |
| | methylpyrimidin-4-yl)methyl)-1H-pyrazolo[3,4-b]pyrazine-3- | |
| | carboxamide | |
| 74 | 1-((5-fluoro-2-methoxy-6-methylpyridin-3-yl)methyl)-N-((1- | 401.16 |
| | hydroxycyclopropyl)methyl)-6-methyl-1H-pyrazolo[3,4-b]pyrazine-3- | |
| | carboxamide | |
| 75 | 1-((5-fluoro-2-methoxy-6-methylpyridin-3-yl)methyl)-N-((1- | 417.13 |
| | hydroxycyclopropyl)methyl)-6-methoxy-1H-pyrazolo[3,4-b]pyrazine-3- | |
| | carboxamide | |
| 76 | 1-((5-fluoro-2-methoxypyridin-3-yl)methyl)-N-((1- | 387.13 |
| | hydroxycyclopropyl)methyl)-6-methyl-1H-pyrazolo[3,4-b]pyrazine-3- | |
| | carboxamide | |
| 77 | 1-((5-fluoro-2-methoxypyridin-3-yl)methyl)-N-((1- | 403.12 |
| | hydroxycyclopropyl)methyl)-6-methoxy-1H-pyrazolo[3,4-b]pyrazine-3- | |
| | carboxamide | |
| 78 | N-((1-hydroxycyclopropyl)methyl)-1-((2-methoxy-5- | 437.16 |
| | (trifluoromethyl)pyridin-3-yl)methyl)-6-methyl-1H-pyrazolo[3,4- | |
| | b]pyrazine-3-carboxamide | |
| 79 | N-((1-hydroxycyclopropyl)methyl)-6-methoxy-1-((2-methoxy-5- | 453.12 |
| | (trifluoromethyl)pyridin-3-yl)methyl)-1H-pyrazolo[3,4-b]pyrazine-3- | |
| | carboxamide | |
| 80 | N-((1-hydroxycyclopropyl)methyl)-6-methoxy-1-((6-methoxy-5- | 398.17 |
| | methylpyrimidin-4-yl)methyl)-1H-pyrrolo[3,2-b]pyridine-3- | |
| | carboxamide | |
| 81 | 1-((6-(dimethylamino)-5-methylpyrimidin-4-yl)methyl)-N-((1- | 395.20 |
| | hydroxycyclopropyl)methyl)-6-methyl-1H-pyrrolo[3,2-b]pyridine-3- | |
| | carboxamide | |
| 82 | 1-((6-(dimethylamino)-5-methylpyrimidin-4-yl)methyl)-N-((1- | 411.2 |
| | hydroxycyclopropyl)methyl)-6-methoxy-1H-pyrrolo[3,2-b]pyridine-3- | |
| | carboxamide | |
| 83 | 1-((6-ethoxy-5-methylpyrimidin-4-yl)methyl)-N-((1- | 396.18 |
| | hydroxycyclopropyl)methyl)-6-methyl-1H-pyrrolo[3,2-b]pyridine-3- | |
| | carboxamide | |
| 84 | 1-((6-ethoxy-5-methylpyrimidin-4-yl)methyl)-N-((1- | 412.20 |
| | hydroxycyclopropyl)methyl)-6-methoxy-1H-pyrrolo[3,2-b]pyridine-3- | |
| | carboxamide | |

### Example 85

### Preparation of N-((1-hydroxycyclopropyl)methyl)-1-((6-methoxy-5-methylpyrimidin-4-yl)methyl)-6-methyl-1H-pyrrolo[3,2-b]pyridine-3-carboxamide

### Step 1: ethyl 3-(dimethylamino)-2-(5-methyl-3-nitropyridin-2-yl)acrylate

To a stirring solution of ethyl 2-(5-methyl-3-nitropyridin-2-yl)acetate (8.7 g, 38.8 mmol) in DMF (87 ml) was added DMF-DMA (5 g, 58.2 mmol) at 25-30° C. The reaction mixture was stirred at 80° C. for 16 h. After complete conversion of starting material reaction mixture was cooled to 15° C. The reaction mixture was diluted with water (500 ml) and extracted by ethyl acetate (2×200 ml). Organic layer was separated, washed with water, dried over Na₂SO₄ and evaporated under reduced pressure to get the title product. The crude material was taken for next step without further purification. ESI-MS (m/z): 280.18. (M+H)⁺.

### Step 2: ethyl 6-methyl-1H-pyrrolo[3,2-b]pyridine-3-carboxylate

To a stirring solution of product of step 1 (10 g, 35.8 mmol) in EtOH (200 ml) was added 10% Pd/C (50% wet) (1.9 g, 17.9 mmol) at 25-30° C. in hydrogenation vessel. The reaction mixture was stirred at 25-30° C. for 16 h under hydrogen pressure. After complete conversion of starting material reaction mixture was filtered through celite bad and evaporated under reduced pressure to get the title product. The crude material was taken for next step without further purification. ESI-MS (m/z): 205.10 (M+H)⁺.

### Step 3: ethyl 1-((6-methoxy-5-methylpyrimidin-4-yl)methyl)-6-methyl-1H-pyrrolo[3,2-b]pyridine-3-carboxylate

To a stirring suspension of product of step 2 (1.0 g, 4.90 mmol) and K₂CO₃ (1.0 g, 7.34 mmol) in dry DMF (5 ml) was added 4-(chloromethyl)-6-methoxy-5-methylpyrimidine (1.0 g, 7.88 mmol) at 10-15° C. and the resulting mixture was stirred at 55-60° C. for 3 h. After complete conversion of starting material reaction mixture was diluted with water (20 ml) and extracted by DCM (3×30 ml). Organic layer was separated, washed with water, dried over Na₂SO₄ and evaporated under reduced pressure to get the crude product which was further purified by column chromatography (0-50% Ethyl acetate in Hexane) to get the title product. ESI-MS (m/z): 341.16 (M+H)⁺.

### Step 4: 1-((6-methoxy-5-methylpyrimidin-4-yl)methyl)-6-methyl-1H-pyrrolo[3,2-b]pyridine-3-carboxylic acid

To a stirring solution of product of step 3 (1.0 g, 2.94 mmol) in EtOH (10 ml) was added NaOH (235 mg (5.88 mmol) in 1 ml water). The reaction mixture was stirred at 55-60° C. for 1 h. After complete conversion of starting material reaction mixture was diluted with water (5 ml) and pH was adjusted to 4 by using 5% HCl solution. The precipitate was filtered, washed with water and dried to get the tile product as solid. ESI-MS (m/z): 311.03 (M−H).

### Step 5: N-((1-hydroxycyclopropyl)methyl)-1-((6-methoxy-5-methylpyrimidin-4-yl)methyl)-6-methyl-1H-pyrrolo[3,2-b]pyridine-3-carboxamide

To a stirring solution of product of step 4 (150 mg, 0.48 mmol) in DCM (6 mL) was added EDC.HCl (184 mg, 0.960 mmol) and HOBT (125 mg, 0.816 mmol) at 25-30° C. Reaction mixture was stirred for 10 min. at 25-30° C. To this was added 1-(aminomethyl)cyclopropan-1-ol (63 mg, 0.720 mmol) and stirred for 16 h. After 16 h, reaction mixture was diluted with DCM (10 ml). Reaction mixture was washed with saturated solution of NaHCO₃, dried over Na₂SO₄ and filtered. Removal of the solvent under reduced pressure gave crude product which was further purified by column chromatography (0-5% CH₃OH in Ethyl acetate to get title product as solid. ¹H NMR (DMSO-d₆): 8.92-8.89 (t, J=5.6 Hz, 1H), 8.42 (s, 1H), 8.35 (s, 1H), 8.12 (s, 1H), 7.75 (s, 1H), 5.64 (s, 2H), 5.48 (s, 1H), 3.94 (s, 3H), 3.51-3.50 (d, J=5.6 Hz, 2H), 2.40 (s, 3H), 2.24 (s, 3H), 0.62-0.54 (m, 4H). ESI-MS (m/z): 382.11 (M+H)⁺.

Examples 80-84 were prepared using appropriate starting materials and suitable modifications of the process described in example 85 The following compounds can be prepared by procedure similar to those described above with appropriate variations of reactions, reaction conditions, reagents and quantities of reagents which are within the scope of person skilled in the art.
N-((1-hydroxycyclopropyl)methyl)-5-((6-methoxy-5-methylpyrimidin-4-yl)methyl)-3-(methylthio)-5H-pyrrolo[2,3-b]pyrazine-7-carboxamide;
3-cyclopropyl-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yloxy)-5H-pyrrolo[2,3-b]pyrazine-7-carboxamide;
3-(cyclopropylmethoxy)-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(2-methoxyethoxy)-5H-pyrrolo[2,3-b]pyrazine-7-carboxamide;
5-((6-methoxy-5-methylpyrimidin-4-yl)methyl)-3-methyl-N-((1-(methylthio)cyclopropyl)methyl)-5H-pyrrolo[2,3-b]pyrazine-7-carboxamide;
N-((1-hydroxycyclopropyl)methyl)-5-((6-methoxy-4-methylpyridazin-3-yl)methyl)-3-methyl-5H-pyrrolo[2,3-b]pyrazine-7-carboxamide;
5-((4-(dimethylamino)-6-methyl-1,3,5-triazin-2-yl)methyl)-N-((1-hydroxycyclopropyl)methyl)-3-methyl-5H-pyrrolo[2,3-b]pyrazine-7-carboxamide.

### Biological Evaluation:

### Minimum Inhibitory Concentration (MIC) Determination

To assess the bacterial growth inhibitory effect of the compounds of the invention, MABA assay was performed with log phase culture of Mycobacterium tuberculosis H37Rv. Bacterial culture of optical density 0.6-0.8 was diluted to final density of 0.02 in 7H9 medium. 100 μl of diluted Mycobacterium tuberculosis H37Rv was incubated with the test compounds in microtiter plate. A drug free control containing DMSO also was also prepared. Following incubation of the compound with culture for 7 days at 37° C., 20 μl of 0.02% alamar blue (freshly prepared) was added in each well. The color was allowed to develop for 16 h at 37° C. Blue color in the well indicated no growth whereas pink color signifies growth in the well. MIC value is defined as the lowest concentration that prevents the color change from blue to pink. Fluorescence was measured at excitation at 530 nm and emission at 590 nm. MIC values of the selected compounds are shown in Table 2.

TABLE 2

| Example | Mtb MIC (μM) |
|---|---|
| 1 | 1.25 |
| 3 | ≤0.625 |
| 10 | 5 |
| 11 | 0.5 |
| 12 | 0.25 |
| 13 | 0.25 |
| 85 | ≤0.0625 |

### Pharmacokinetics of the Compounds

Balb/c mice were obtained from Zydus Research Centre, Ahmedabad India, AAALAC Accreditation. All animals were housed in temperature-control rooms with appropriate light/dark cycles. The animals were overnight fasted before oral gavage dosing but were given access to water ad libitum. Food was provided 4 h after dosing. All animal studies were conducted according to protocols reviewed and approved by the Institutional Animal Care and Ethics Committee at the Zydus Research Centre. Compounds were dosed as a solution in 10% NMP and 10% solutol in Normal Saline (Intravenous (IV), 1 mg/kg) and homogenous suspension (Oral (PO), 3 mg/kg) in 0.5% Tween 80 in 0.5% methyl cellulose as a single dose in mice. For mouse composite PK profile was generated. Blood samples were collected at 0.08 (IV only), 0.25, 0.5, 1, 2, 4, 6, 7.5, and 24 h post-dose in Na-heparin coated micro centrifuge tubes. Blood samples were centrifuged to separate plasma which were then stored at −70° C. until analysis. PK parameters were calculated by non-compartmental analysis using Phoenix (Pharsight Corp., Mountain View, Calif.). A model was selected based on the vascular (IV bolus) or extravascular (PO) routes of administration. For the PO route, concentration at time zero was assumed to be zero. Plasma concentrations below the limit of quantitation were treated as zero concentration for the purpose of calculating the mean plasma concentration values. Pharmacokinetic parameters such as maximum plasma concentration (Cmax), area under the curve of concentration over the time (AUC (0-t)) and absolute oral bioavailability (F (%)) of the selected compounds are shown in Table 3.

TABLE 3

| Example No. | Cmax (ng/mL) | AUC (0-t) (ng · h/mL) | F(%) |
|---|---|---|---|
| 1 | 1726 | 2849 | 100 |
| 3 | 2723 | 2814 | 100 |
| 9 | 1570 | 2158 | 78 |
| 10 | 4004 | 5129 | 100 |
| 11 | 1661 | 2567 | 100 |
| 12 | 1716 | 2919 | 100 |
| 13 | 1317 | 2152 | 100 |
| 14 | 1605 | 1682 | 90 |
| 85 | 1467 | 2176 | 94 |

The novel compounds of the present invention can be formulated into suitable pharmaceutically acceptable compositions by combining with suitable excipients by techniques and processes and concentrations as are well known. Pharmaceutical composition comprising the compounds of the present invention may comprise a suitable binder, suitable bulking agent &/or diluent and any other suitable agents as may be necessary.

Novel compounds of the present invention can be used for the treatment of mammalian infections such as tuberculosis by administrating therapeutically active and non-toxic amount of compounds of formula (I) or pharmaceutically acceptable compositions thereof.

A method of treating anti-tuberculosis infection in a subject that comprising administering to a patient in need thereof an effective amount of a compound of Formula (I) or its suitable pharmaceutical composition.

The pharmaceutical compositions according to this invention can exist in various forms. In some embodiments, the pharmaceutical composition is in the topical, oral or parenteral administration.

In certain embodiment, a provided combination, or composition thereof, is administered in combination with other therapeutic agents such as isoniazide, rifampin, rifapentine, rifabutin, ethambutol, pyrazinamide, streptomycin, amikacin, levofloxacin, ofloxacin, p-aminosalicylic acid.

1. A compounds of general formula (I), 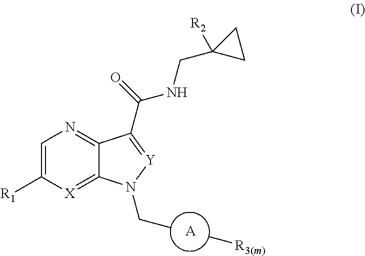
or a pharmaceutically acceptable salt thereof, wherein,
A represents an optionally substituted 6-membered ring systems as given below: 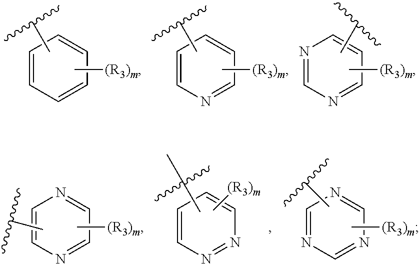
X and Y independently represents either CH or N; provided that X and Y both can not be CH together;
R₁ is independently selected from the group comprising of hydrogen, halo, haloalkyl cyano, —OR₄, —S(O)ₚR₄, —NR₄R₅, —COOR₄, optionally substituted (C₁-C₆)alkyl, (C₃-C₆)cycloalkyl, and heterocyclyl derivatives;
with a proviso that when R₁ is H, then A represents cyclic ring other than phenyl and pyridine;
R₂ is halogen, —OR₆, or —S(O)ₚR₆;
R₃, at each occurrence, is independently selected from hydrogen, halo, cyano, haloalkyl, —NR₄R₅, S(O)ₚR₄, —OR₄, and optionally substituted (C₁-C₆)alkyl;
each of R₄ and R₅, are at each occurrence, are independently selected from hydrogen, optionally substituted (C₁-C₆)alkyl, (C₃-C₆)cycloalkyl, cycloalkanyl alkyl, heterocyclyl, haloalkyl; alternatively, R₄ and R₅ together with the N atom to which they are attached may form a 5-8 membered cyclic ring which may contain additional heteroatoms chosen from O, —S(O)ₚ and —NR₆;
R₆ is selected from H and (C₁-C₆)alkyl;
the (C₁-C₆) alkyl chain as used herein before, may further substituted with hydrogen, hydroxy, cyano, halo, haloalkyl, oxo, (C₁-C₆)alkyl, (C₃-C₆)cycloalkyl, heterocyclyl, alkylsulfonyloxy, —COOR₄, —OR₄, —S(O)ₚR₄, —NR₄R₅, —CONR₄R₅, —N(R₄)COR₅, —SO₂NR₄R₅, —N(R₄)SO₂R₅ derivatives; wherein, R₄ and R₅ are as defined earlier;
p represents integers from 0-2; and m represents an integers from 1-5.

2. The compound of formula (I) as claimed in claim 1 wherein A is selected from following groups:

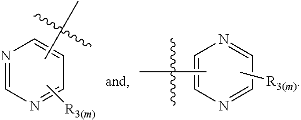

3. The compound of formula (I) as claimed in claim 1 wherein R₁ is selected from —CH₃, —OCH₃, and halogen.

4. The compound of formula (I) as claimed in claim 1 wherein R₂ is a hydroxy group.

5. The compound of formula (I) as claimed in claim 1 wherein the R₃ group is independently selected from —CH₃, —OR₄, —SCH₃, —N(CH₃)₂, and halogen.

6. A compounds selected from
N-((1-hydroxycyclopropyl)methyl)-1-((6-methoxy-5-methylpyrimidin-4-yl)methyl)-6-methyl-1H-pyrazolo[4,3-b]pyridine-3-carboxamide;
1-((6-methoxy-5-methylpyrimidin-4-yl)methyl)-N-((1-methoxycyclopropyl)methyl)-6-methyl-1H-pyrazolo[4,3-b]pyridine-3-carboxamide;
1-((6-ethoxy-5-methylpyrimidin-4-yl)methyl)-N-((1-hydroxycyclopropyl)methyl)-6-methyl-1H-pyrazolo[4,3-b]pyridine-3-carboxamide;
N-((1-hydroxycyclopropyl)methyl)-6-methyl-1-((5-methyl-6-(methylthio)pyrimidin-4-yl)methyl)-1H-pyrazolo[4,3-b]pyridine-3-carboxamide;
N-((1-hydroxycyclopropyl)methyl)-1-((6-(2-methoxyethoxy)-5-methylpyrimidin-4-yl)methyl)-6-methyl-1H-pyrazolo[4,3-b]pyridine-3-carboxamide;
1-((6-(dimethylamino)-5-methylpyrimidin-4-yl)methyl)-N-((1-hydroxycyclopropyl) methyl)-6-methyl-1H-pyrazolo[4,3-b]pyridine-3-carboxamide;
N-((1-hydroxycyclopropyl)methyl)-6-methyl-1-((5-methyl-6-morpholinopyrimidin-4-yl)methyl)-1H-pyrazolo[4,3-b]pyridine-3-carboxamide;
1-((6-(cyclopropylmethoxy)-5-methylpyrimidin-4-yl)methyl)-N-((1-hydroxycyclopropyl) methyl)-6-methyl-1H-pyrazolo[4,3-b]pyridine-3-carboxamide;
N-((1-hydroxycyclopropyl)methyl)-1-((2-methoxy-6-methylpyridin-3-yl)methyl)-6-methyl-1H-pyrazolo[4,3-b]pyridine-3-carboxamide;
1-((6-methoxy-5-methylpyrimidin-4-yl)methyl)-N-(2-methoxyethyl)-6-methyl-1H-pyrazolo[4,3-b]pyridine-3-carboxamide;
N-((1-hydroxycyclopropyl)methyl)-5-((6-methoxy-5-methylpyrimidin-4-yl)methyl)-3-methyl-5H-pyrrolo[2,3-b]pyrazine-7-carboxamide;
N-((1-hydroxycyclopropyl)methyl)-3-methoxy-5-((6-methoxy-5-methylpyrimidin-4-yl)methyl)-5H-pyrrolo[2,3-b]pyrazine-7-carboxamide;
5-((6-ethoxy-5-methylpyrimidin-4-yl)methyl)-N-((1-hydroxycyclopropyl)methyl)-3-methyl-5H-pyrrolo[2,3-b]pyrazine-7-carboxamide;
N-((1-hydroxycyclopropyl)methyl)-1-((6-methoxy-5-methylpyrimidin-4-yl)methyl)-6-methyl-1H-pyrazolo[3,4-b]pyrazine-3-carboxamide;
N-((1-hydroxycyclopropyl)methyl)-6-methoxy-1-((6-methoxy-5-methylpyrimidin-4-yl)methyl)-1H-pyrazolo[3,4-b]pyrazine-3-carboxamide;
N-((1-hydroxycyclopropyl)methyl)-6-methyl-1-((5-methyl-6-(oxetan-3-yloxy)pyrimidin-4-yl)methyl)-1H-pyrazolo[4,3-b]pyridine-3-carboxamide;
N-((1-hydroxycyclopropyl)methyl)-6-methoxy-1-((6-methoxy-5-methylpyrimidin-4-yl)methyl)-1H-pyrazolo[4,3-b]pyridine-3-carboxamide;
1-((6-ethoxy-5-methylpyrimidin-4-yl)methyl)-N-((1-hydroxycyclopropyl)methyl)-6-methoxy-1H-pyrazolo[4,3-b]pyridine-3-carboxamide;
1-((6-(dimethylamino)-5-methylpyrimidin-4-yl)methyl)-N-((1-hydroxycyclopropyl) methyl)-6-methoxy-1H-pyrazolo[4,3-b]pyridine-3-carboxamide;
1-((6-(ethylthio)-5-methylpyrimidin-4-yl)methyl)-N-((1-hydroxycyclopropyl)methyl)-6-methyl-1H-pyrazolo[4,3-b]pyridine-3-carboxamide;
1-((6-(difluoromethoxy)-5-methylpyrimidin-4-yl)methyl)-N-((1-hydroxycyclopropyl) methyl)-6-methyl-1H-pyrazolo[4,3-b]pyridine-3-carboxamide;
N-((1-fluorocyclopropyl)methyl)-1-((6-methoxy-5-methylpyrimidin-4-yl)methyl)-6-methyl-1H-pyrazolo[4,3-b]pyridine-3-carboxamide;
1-((5-fluoro-2,6-dimethylpyridin-3-yl)methyl)-N-((1-hydroxycyclopropyl)methyl)-6-methyl-1H-pyrazolo[4,3-b]pyridine-3-carboxamide;
1-((3,5-dimethylpyrazin-2-yl)methyl)-N-((1-hydroxycyclopropyl)methyl)-6-methyl-1H-pyrazolo[4,3-b]pyridine-3-carboxamide;
6-chloro-N-((1-hydroxycyclopropyl)methyl)-1-((6-methoxy-5-methylpyrimidin-4-yl)methyl)-1H-pyrazolo[4,3-b]pyridine-3-carboxamide;
1-((5-fluoro-2-methoxy-6-methylpyridin-3-yl)methyl)-N-((1-hydroxycyclopropyl) methyl)-6-methyl-1H-pyrazolo[4,3-b]pyridine-3-carboxamide;
N-((1-hydroxycyclopropyl)methyl)-1-((2-methoxy-5-(trifluoromethyl)pyridin-3-yl)methyl)-6-methyl-1H-pyrazolo[4,3-b]pyridine-3-carboxamide;
1-((5-fluoro-2-methoxypyridin-3-yl)methyl)-N-((1-hydroxycyclopropyl)methyl)-6-methyl-1H-pyrazolo[4,3-b]pyridine-3-carboxamide;
1-(2,4-dimethylbenzyl)-N-((1-hydroxycyclopropyl)methyl)-6-methyl-1H-pyrazolo[4,3-b]pyridine-3-carboxamide;
5-((6-ethoxy-5-methylpyrimidin-4-yl)methyl)-N-((1-hydroxycyclopropyl)methyl)-3-methoxy-5H-pyrrolo[2,3-b]pyrazine-7-carboxamide;
5-((6-(dimethylamino)-5-methylpyrimidin-4-yl)methyl)-N-((1-hydroxycyclopropyl) methyl)-3-methyl-5H-pyrrolo[2,3-b]pyrazine-7-carboxamide;
5-((6-(dimethylamino)-5-methylpyrimidin-4-yl)methyl)-N-((1-hydroxycyclopropyl) methyl)-3-methoxy-5H-pyrrolo[2,3-b]pyrazine-7-carboxamide;
N-((1-hydroxycyclopropyl)methyl)-3-methoxy-5-((5-methyl-6-(methylthio)pyrimidin-4-yl)methyl)-5H-pyrrolo[2,3-b]pyrazine-7-carboxamide
N-((1-hydroxycyclopropyl)methyl)-3-methyl-5-((5-methyl-6-(methylthio)pyrimidin-4-yl)methyl)-5H-pyrrolo[2,3-b]pyrazine-7-carboxamide;
5-((6-(ethylthio)-5-methylpyrimidin-4-yl)methyl)-N-((1-hydroxycyclopropyl)methyl)-3-methyl-5H-pyrrolo[2,3-b]pyrazine-7-carboxamide
5-((6-(ethylthio)-5-methylpyrimidin-4-yl)methyl)-N-((1-hydroxycyclopropyl)methyl)-3-methoxy-5H-pyrrolo[2,3-b]pyrazine-7-carboxamide;
5-((6-(difluoromethoxy)-5-methylpyrimidin-4-yl)methyl)-N-((1-hydroxycyclopropyl) methyl)-3-methyl-5H-pyrrolo[2,3-b]pyrazine-7-carboxamide;
5-((6-(difluoromethoxy)-5-methylpyrimidin-4-yl)methyl)-N-((1-hydroxycyclopropyl) methyl)-3-methoxy-5H-pyrrolo[2,3-b]pyrazine-7-carboxamide;
N-((1-fluorocyclopropyl)methyl)-5-((6-methoxy-5-methylpyrimidin-4-yl)methyl)-3-methyl-5H-pyrrolo[2,3-b]pyrazine-7-carboxamide;
N-((1-fluorocyclopropyl)methyl)-3-methoxy-5-((6-methoxy-5-methylpyrimidin-4-yl)methyl)-5H-pyrrolo[2,3-b]pyrazine-7-carboxamide;
N-((1-hydroxycyclopropyl)methyl)-5-((2-methoxy-6-methylpyridin-3-yl)methyl)-3-methyl-5H-pyrrolo[2,3-b]pyrazine-7-carboxamide;
N-((1-hydroxycyclopropyl)methyl)-3-methoxy-5-((2-methoxy-6-methylpyridin-3-yl)methyl)-5H-pyrrolo[2,3-b]pyrazine-7-carboxamide;
5-((5-fluoro-2,6-dimethylpyridin-3-yl)methyl)-N-((1-hydroxycyclopropyl)methyl)-3-methyl-5H-pyrrolo[2,3-b]pyrazine-7-carboxamide;
5-((5-fluoro-2,6-dimethylpyridin-3-yl)methyl)-N-((1-hydroxycyclopropyl)methyl)-3-methoxy-5H-pyrrolo[2,3-b]pyrazine-7-carboxamide;
5-((3,5-dimethylpyrazin-2-yl)methyl)-N-((1-hydroxycyclopropyl)methyl)-3-methyl-5H-pyrrolo[2,3-b]pyrazine-7-carboxamide;
5-((3,5-dimethylpyrazin-2-yl)methyl)-N-((1-hydroxycyclopropyl)methyl)-3-methoxy-5H-pyrrolo[2,3-b]pyrazine-7-carboxamide;
N-((1-hydroxycyclopropyl)methyl)-5-((6-methoxy-5-methylpyrimidin-4-yl)methyl)-5H-pyrrolo[2,3-b]pyrazine-7-carboxamide;
5-((5-fluoro-2-methoxy-6-methylpyridin-3-yl)methyl)-N-((1-hydroxycyclopropyl) methyl)-3-methyl-5H-pyrrolo[2,3-b]pyrazine-7-carboxamide;
5-((5-fluoro-2-methoxy-6-methylpyridin-3-yl)methyl)-N-((1-hydroxycyclopropyl) methyl)-3-methoxy-5H-pyrrolo[2,3-b]pyrazine-7-carboxamide;
5-((5-fluoro-2-methoxypyridin-3-yl)methyl)-N-((1-hydroxycyclopropyl)methyl)-3-methyl-5H-pyrrolo[2,3-b]pyrazine-7-carboxamide;
5-((5-fluoro-2-methoxypyridin-3-yl)methyl)-N-((1-hydroxycyclopropyl)methyl)-3-methoxy-5H-pyrrolo[2,3-b]pyrazine-7-carboxamide;
N-((1-hydroxycyclopropyl)methyl)-5-((2-methoxy-5-(trifluoromethyl)pyridin-3-yl)methyl)-3-methyl-5H-pyrrolo[2,3-b]pyrazine-7-carboxamide;
N-((1-hydroxycyclopropyl)methyl)-3-methoxy-5-((2-methoxy-5-(trifluoromethyl)pyridin-3-yl)methyl)-5H-pyrrolo[2,3-b]pyrazine-7-carboxamide;
3-(dimethylamino)-N-((1-hydroxycyclopropyl)methyl)-5-((6-methoxy-5-methylpyrimidin-4-yl)methyl)-5H-pyrrolo[2,3-b]pyrazine-7-carboxamide;
3-cyano-N-((1-hydroxycyclopropyl)methyl)-5-((6-methoxy-5-methylpyrimidin-4-yl)methyl)-5H-pyrrolo[2,3-b]pyrazine-7-carboxamide;
3-ethoxy-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-3-isopropoxy-5-((6-methoxy-5-methylpyrimidin-4-yl)methyl)-5H-pyrrolo[2,3-b]pyrazine-7-carboxamide;
N-((1-hydroxycyclopropyl)methyl)-6-methyl-1-((5-methyl-6-(methylthio)pyrimidin-4-yl)methyl)-1H-pyrazolo[3,4-b]pyrazine-3-carboxamide;
N-((1-hydroxycyclopropyl)methyl)-6-methoxy-1-((5-methyl-6-(methylthio)pyrimidin-4-yl)methyl)-1H-pyrazolo[3,4-b]pyrazine-3-carboxamide;
1-((6-(dimethylamino)-5-methylpyrimidin-4-yl)methyl)-N-((1-hydroxycyclopropyl)methyl)-6-methoxy-1H-pyrazolo[3,4-b]pyrazine-3-carboxamide;
1-((6-(dimethylamino)-5-methylpyrimidin-4-yl)methyl)-N-((1-hydroxycyclopropyl)methyl)-6-methyl-1H-pyrazolo[3,4-b]pyrazine-3-carboxamide;
1-((6-ethoxy-5-methylpyrimidin-4-yl)methyl)-N-((1-hydroxycyclopropyl)methyl)-6-methyl-1H-pyrazolo[3,4-b]pyrazine-3-carboxamide;
1-((6-ethoxy-5-methylpyrimidin-4-yl)methyl)-N-((1-hydroxycyclopropyl)methyl)-6-methoxy-1H-pyrazolo[3,4-b]pyrazine-3-carboxamide;
1-((6-(difluoromethoxy)-5-methylpyrimidin-4-yl)methyl)-N-((1-hydroxycyclopropyl)methyl)-6-methyl-1H-pyrazolo[3,4-b]pyrazine-3-carboxamide;
1-((6-(difluoromethoxy)-5-methylpyrimidin-4-yl)methyl)-N-((1-hydroxycyclopropyl)methyl)-6-methoxy-1H-pyrazolo[3,4-b]pyrazine-3-carboxamide;
N-((1-hydroxycyclopropyl)methyl)-1-((2-methoxy-6-methylpyridin-3-yl)methyl)-6-methyl-1H-pyrazolo[3,4-b]pyrazine-3-carboxamide;
N-((1-hydroxycyclopropyl)methyl)-6-methoxy-1-((2-methoxy-6-methylpyridin-3-yl)methyl)-1H-pyrazolo[3,4-b]pyrazine-3-carboxamide;
1-((5-fluoro-2,6-dimethylpyridin-3-yl)methyl)-N-((1-hydroxycyclopropyl)methyl)-6-methyl-1H-pyrazolo[3,4-b]pyrazine-3-carboxamide;
1-((5-fluoro-2,6-dimethylpyridin-3-yl)methyl)-N-((1-hydroxycyclopropyl)methyl)-6-methoxy-1H-pyrazolo[3,4-b]pyrazine-3-carboxamide;
1-((3,5-dimethylpyrazin-2-yl)methyl)-N-((1-hydroxycyclopropyl)methyl)-6-methyl-1H-pyrazolo[3,4-b]pyrazine-3-carboxamide;
1-((3,5-dimethylpyrazin-2-yl)methyl)-N-((1-hydroxycyclopropyl)methyl)-6-methoxy-1H-pyrazolo[3,4-b]pyrazine-3-carboxamide;
N-((1-fluorocyclopropyl)methyl)-1-((6-methoxy-5-methylpyrimidin-4-yl)methyl)-6-methyl-1H-pyrazolo[3,4-b]pyrazine-3-carboxamide;
N-((1-fluorocyclopropyl)methyl)-6-methoxy-1-((6-methoxy-5-methylpyrimidin-4-yl)methyl)-1H-pyrazolo[3,4-b]pyrazine-3-carboxamide;
1-((5-fluoro-2-methoxy-6-methylpyridin-3-yl)methyl)-N-((1-hydroxycyclopropyl) methyl)-6-methyl-1H-pyrazolo[3,4-b]pyrazine-3-carboxamide;
1-((5-fluoro-2-methoxy-6-methylpyridin-3-yl)methyl)-N-((1-hydroxycyclopropyl) methyl)-6-methoxy-1H-pyrazolo[3,4-b]pyrazine-3-carboxamide;
1-((5-fluoro-2-methoxypyridin-3-yl)methyl)-N-((1-hydroxycyclopropyl)methyl)-6-methyl-1H-pyrazolo[3,4-b]pyrazine-3-carboxamide;
1-((5-fluoro-2-methoxypyridin-3-yl)methyl)-N-((1-hydroxycyclopropyl)methyl)-6-methoxy-1H-pyrazolo[3,4-b]pyrazine-3-carboxamide;
N-((1-hydroxycyclopropyl)methyl)-1-((2-methoxy-5-(trifluoromethyl)pyridin-3-yl)methyl)-6-methyl-1H-pyrazolo[3,4-b]pyrazine-3-carboxamide;
N-((1-hydroxycyclopropyl)methyl)-6-methoxy-1-((2-methoxy-5-(trifluoromethyl)pyridin-3-yl)methyl)-1H-pyrazolo[3,4-b]pyrazine-3-carboxamide;
N-((1-hydroxycyclopropyl)methyl)-1-((6-methoxy-5-methylpyrimidin-4-yl)methyl)-6-methyl-1H-pyrrolo[3,2-b]pyridine-3-carboxamide;
N-((1-hydroxycyclopropyl)methyl)-6-methoxy-1-((6-methoxy-5-methylpyrimidin-4-yl)methyl)-1H-pyrrolo[3,2-b]pyridine-3-carboxamide;
1-((6-(dimethylamino)-5-methylpyrimidin-4-yl)methyl)-N-((1-hydroxycyclopropyl)methyl)-6-methyl-1H-pyrrolo[3,2-b]pyridine-3-carboxamide;
1-((6-(dimethylamino)-5-methylpyrimidin-4-yl)methyl)-N-((1-hydroxycyclopropyl)methyl)-6-methoxy-1H-pyrrolo[3,2-b]pyridine-3-carboxamide;
1-((6-ethoxy-5-methylpyrimidin-4-yl)methyl)-N-((1-hydroxycyclopropyl)methyl)-6-methyl-1H-pyrrolo[3,2-b]pyridine-3-carboxamide;
1-((6-ethoxy-5-methylpyrimidin-4-yl)methyl)-N-((1-hydroxycyclopropyl)methyl)-6-methoxy-1H-pyrrolo[3,2-b]pyridine-3-carboxamide;
N-((1-hydroxycyclopropyl)methyl)-5-((6-methoxy-5-methylpyrimidin-4-yl)methyl)-3-(methylthio)-5H-pyrrolo[2,3-b]pyrazine-7-carboxamide;
3-cyclopropyl-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(oxetan-3-yloxy)-5H-pyrrolo[2,3-b]pyrazine-7-carboxamide;
3-(cyclopropylmethoxy)-N-((1-hydroxycyclopropyl)methyl)-5-((6-methoxy-5-methylpyrimidin-4-yl)methyl)-5H-pyrrolo[2,3-b]pyrazine-7-carboxamide;
N-((1-hydroxycyclopropyl)methyl)-5-((6-methoxy-5-methylpyrimidin-4-yl)methyl)-3-(2-methoxyethoxy)-5H-pyrrolo[2,3-b]pyrazine-7-carboxamide;
5-((6-methoxy-5-methylpyrimidin-4-yl)methyl)-3-methyl-N-((1-(methylthio)cyclopropyl)methyl)-5H-pyrrolo[2,3-b]pyrazine-7-carboxamide;
N-((1-hydroxycyclopropyl)methyl)-5-((6-methoxy-4-methylpyridazin-3-yl)methyl)-3-methyl-5H-pyrrolo[2,3-b]pyrazine-7-carboxamide; and
5-((4-(dimethylamino)-6-methyl-1,3,5-triazin-2-yl)methyl)-N-((1-hydroxycyclopropyl)methyl)-3-methyl-5H-pyrrolo[2,3-b]pyrazine-7-carboxamide; and
the pharmaceutically acceptable salts of any of the foregoing.

7. A pharmaceutical composition comprising the compound or salt of claim 1 and one or more pharmaceutically acceptable carriers, diluents, or excipients.

8. A pharmaceutical composition comprising an amount therapeutically effective for the prevention or treatment of a tuberculosis infection of the compound or salt of claim 1.

9. A method of treating a tuberculosis infection comprising administering to a patient in need thereof an effective amount of a compound or salt claim 1.

10. The Compound or salt of claim 1 in combination with one or more suitable pharmaceutically active agents selected from group consisting of isoniazide, rifampin, rifapentine, rifabutin, ethambutol, pyrazinamide, streptomycin, amikacin, levofloxacin, ofloxacin, and p-aminosalicylic acid.

11. A pharmaceutical composition comprising the compound or salt of claim 6 and one or more pharmaceutically acceptable carriers, diluents, or excipients.

12. A pharmaceutical composition comprising an amount therapeutically effective for the prevention or treatment of a tuberculosis infection of the compound or salt of claim 6.

13. A method of treating a tuberculosis infection comprising administering to a patient in need thereof an effective amount of a compound or salt claim 6.

14. The Compound or salt of claim 6 in combination with one or more suitable pharmaceutically active agents selected from group consisting of isoniazide, rifampin, rifapentine, rifabutin, ethambutol, pyrazinamide, streptomycin, amikacin, levofloxacin, ofloxacin, and p-aminosalicylic acid.
